SEQUENCE LISTING (11 sequences)

SEQ ID NO: 1 - DNA, 975 bp, Clostridium acetobutylicum
atgaaatttggattattttttcttaattttataaatagtacaactattcaagaacagtcaatagcaagaatgcaggagattacagagtatgttgataagctaaattttgagcagattcttgtatgtgaaaatcatttttcagataatggtgttgtaggtgctcctttaactgttagtggttttttattaggacttacagaaaaaattaagataggttcattaaatcatgtaattactacacatcatccagttagaatagcagaagaggcttgccttttagatcaactttctgaaggaagatttatattaggttttagtgattgtgaaagaaaagatgagatgcacttttttaatagacctgaacaatatcaacaacaactttttgaagagtgctatgatattataaatgacgcattaactacaggatattgtaatccaaatggagatttttataattttcctaaaatttcagtaaatccacatgcttatactcagaatggtcctagaaagtatgttacagcaacttcttgtcatgtagttgaatgggcagctaagaagggtataccattaatttttaaatgggatgatagtaatgaagtaaaacatgagtatgctaagagatatcaagcaatagctggtgaatatggagttgatcttgcagaaattgatcatcaattaatgatattagttaattattcagaggattctgaaaaagctaaggaagagacaagagcatttataagtgattatattttagctatgcaccctaatgaaaattttgaaaaaaaacttgaggaaataataactgaaaattcagttggtgattatatggagtgcacaactgctgcaaaacttgcaatggaaaaatgtggagctaaaggtattcttttatcttttgaaagtatgtcagattttacacatcagattaatgcaatagatatagtaaatgataatattaagaaatatcatatgtaa

SEQ ID NO: 2 - protein, 360 aa, Clostridium acetobutylicum
MKFGNFLLTYQPPQFSQTEVMKRLVKLGRISEECGFDTVWLLEHHFTEFGLLGNPYVAAAYLLGATKKLNVGTAAIVLPTAHPVRQLEEVNLLDQMSKGRFRFGICRGLYNKDFRVFGTDMNNSRALMECWYKLIRNGMTEGYMEADNEHIKFHKVKVLPTAYSQGGAPIYVVAESASTTEWAAQHGLPMILSWIINTNEKKAQIELYNEVAQEYGHDIHNIDHCLSYITSVDHDSMKAKEICRNFLGHWYDSYVNATTIFDDSDKTKGYDFNKGQWRDFVLKGHKNTNRRVDYSYEINPVGTPQECIDIIQTDIDATGISNICCGFEANGTVDEIISSMKLFQSDVMPFLKEKQRSLLY

SEQ ID NO: 3 - DNA, 975 bp, Clostridium acetobutylicum; identical to SEQ ID NO: 1

SEQ ID NO: 4 - protein, 324 aa, Clostridium acetobutylicum
MKFGLFFLNFINSTTIQEQSIARMQEITEYVDKLNFEQILVCENHFSDNGVVGAPLTVSGFLLGLTEKIKIGSLNHVITTHHPVRIAEEACLLDQLSEGRFILGFSDCERKDEMHFFNRPEQYQQQLFEECYDIINDALTTGYCNPNGDFYNFPKISVNPHAYTQNGPRKYVTATSCHVVEWAAKKGIPLIFKWDDSNEVKHEYAKRYQAIAGEYGVDLAEIDHQLMILVNYSEDSEKAKEETRAFISDYILAMHPNENFEKKLEEIITENSVGDYMECTTAAKLAMEKCGAKGILLSFESMSDFTHQINAIDIVNDNIKKYHM

SEQ ID NO: 5 - DNA, 1443 bp, Clostridium acetobutylicum
atgaataaaaagatatcatttattataaatggaagagttgaaatatttcctgagtcagatgatttagtacaatctataaattttggtgataattctgttcatcttccagtacttaatgattcacaggttaagaatattatagattataatgagaataatgagcttcagcttcataatattataaattttctttatacagtaggacagagatggaagaatgaggagtatagcagaagaagaacttatataagagatcttaagagatatatgggttatagtgaggaaatggcaaaattagaagctaattggatttcaatgatattatgttctaagggaggtttatatgatttagttaaaaatgaattaggaagtagacatattatggatgaatggttacctcaagatgaatcatatataagagcatttccaaaaggtaaaagtgtacatcttttaacaggaaatgttcctttaagtggagtactttcaattttaagagctatacttactaaaaatcagtgcattataaagacatctagtactgatccatttacagcaaatgctttagcacttagttttatagatgttgatcctcatcatccagtaactagatctttaagtgttgtatattggcaacatcaaggtgatatttcacttgctaaagaaataatgcaacatgcagatgttgtagttgcttggggaggtgaagatgcaattaattgggctgtaaagcacgcacctccagatatagatgttatgaaatttggacctaaaaagtctttttgtattatagataatccagtagatttagttagtgctgcaacaggtgctgcacatgatgtatgcttttatgatcagcaggcttgtttttcaactcaaaatatatattatatgggatcacattatgaagaatttaaacttgcattaattgaaaaacttaatttatatgctcatatacttccaaatacaaagaaagattttgatgaaaaggcagcttatagtttagttcagaaagaatgtttatttgcaggacttaaagtagaagttgatgtacatcaaagatggatggttattgaatcaaatgctggtgtagaattaaatcagccacttggaagatgcgtttatttacatcatgtagataatatagagcaaattttaccttatgttagaaagaataaaactcaaacaatatctgtatttccatgggaagcagctttaaagtatagagatcttttagcacttaaaggtgctgaaagaattgttgaggcaggaatgaataatatatttagagtaggtggtgctcatgatggaatgaggcctttacagagacttgttacttatataagtcatgaaagaccaagtcattatacagcaaaagatgtagctgtagagattgagcaaactagatttttagaagaagataagtttttagtatttgttccttaa

SEQ ID NO: 6 - protein, 480 aa, Clostridium acetobutylicum
MNKKISFIINGRVEIFPESDDLVQSINFGDNSVHLPVLNDSQVKNIIDYNENNELQLHNIINFLYTVGQRWKNEEYSRRRTYIRDLKRYMGYSEEMAKLEANWISMILCSKGGLYDLVKNELGSRHIMDEWLPQDESYIRAFPKGKSVHLLTGNVPLSGVLSILRAILTKNQCIIKTSSTDPFTANALALSFIDVDPHHPVTRSLSVVYWQHQGDISLAKEIMQHADVVVAWGGEDAINWAVKHAPPDIDVMKFGPKKSFCIIDNPVDLVSAATGAAHDVCFYDQQACFSTQNIYYMGSHYEEFKLALIEKLNLYAHILPNTKKDFDEKAAYSLVQKECLFAGLKVEVDVHQRWMVIESNAGVELNQPLGRCVYLHHVDNIEQILPYVRKNKTQTISVFPWEAALKYRDLLALKGAERIVEAGMNNIFRVGGAHDGMRPLQRLVTYISHERPSHYTAKDVAVEIEQTRFLEEDKFLVFVP

SEQ ID NO: 7 - DNA, 924 bp, Clostridium acetobutylicum
atggaaaataaaagtagatataagacaatagatcatgttatttgtgtagaggagaatagaaagatacatgtttgggaaactttacctaaagaaaattcaccaaaaagaaaaaatacacttattatagcatctggatttgctagaagaatggatcattttgctggtttagctgaatatttatctcaaaatggatttcatgtaattagatatgattcattacatcatgttggtttaagttcaggaactatagatgaatttacaatgtcaattggtaagcagagtttacttgcagtagttgattggttaaatactagaaaaataaataatcttggaatgttagctagttcattatctgcaagaatagcttatgcaagtctttcagagattaatgtatcttttcttataacagctgttggtgtagttaatttaagatatactttagaaagagcacttggatttgattatcttagccttcctattgatgaattaccagataatcttgattttgagggacataagttaggtgctgaagtatttgcaagagattgctttgattcaggatgggaagatcttacatctactataaatagtatgatgcacttagatattccttttatagcttttacagcaaataatgatgattgggttaaacaagatgaggtaattactcttctttctagtataagaagtcatcagtgtaaaatatattcacttttaggttctagtcatgatcttggagaaaatttagttgtattaagaaatttttatcaatcagttacaaaggctgcaattgctatggataatggttgccttgatatagatgtagatattatagaaccatcttttgagcatttaactattgcagctgttaatgaaagaagaatgaaaatagaaatagagaatcaagtaattagtttaagttaa

SEQ ID NO: 8 - protein, 307 aa, Clostridium acetobutylicum
MENKSRYKTIDHVICVEENRKIHVWETLPKENSPKRKNTLIIASGFARRMDHFAGLAEYLSQNGFHVIRYDSLHHVGLSSGTIDEFTMSIGKQSLLAVVDWLNTRKINNLGMLASSLSARIAYASLSEINVSFLITAVGVVNLRYTLERALGFDYLSLPIDELPDNLDFEGHKLGAEVFARDCFDSGWEDLTSTINSMMHLDIPFIAFTANNDDWVKQDEVITLLSSIRSHQCKIYSLLGSSHDLGENLVVLRNFYQSVTKAAIAMDNGCLDIDVDIIEPSFEHLTIAAVNERRMKIEIENQVISLS

SEQ ID NO: 9 - DNA, 1113 bp, Clostridium acetobutylicum
atgacatcttatgttgataaacaagaaataactgcaagttcagagattgatgatttaatatttagttcagatcctcttgtatggtcttatgatgaacaggaaaagattagaaaaaagttagttcttgatgcttttagacatcattataaacattgtcaagagtatagacattattgccaggcacataaagtagatgataatataacagaaattgatgatataccagtttttcctacttcagtatttaagtttacaagattacttacttcaaatgaaaatgagattgaatcatggtttacaagttcaggaactaatggtttaaaatctcaagttccaagagatagacttagtatagaaagacttttaggatcagtatcttatggtatgaagtatataggaagttggtttgatcatcaaatggagttagttaatcttggtcctgatagatttaatgctcataatatttggtttaaatatgtaatgtcacttgtagaacttttatatccaacaagttttactgtaacagaagagcatatagattttgttcagactttaaatagtcttgaaagaattaaacatcaaggaaaggatatatgtttaattggttcaccttattttatatatcttttatgcagatatatgaaagataagaatatttcttttagtggagataaatcactttatataataactggaggtggatggaaatcttatgaaaaggagagtttaaaaagaaatgattttaatcatcttttatttgatacttttaatctttcaaatattaatcaaataagagatatttttaatcaggtagaattaaatacatgtttttttgaggatgaaatgcaaagaaaacatgttccaccttgggtatatgcaagggctcttgatccagaaactttaaagcctgttccagatggtatgcctggacttatgtcttatatggatgcttcaagtactagttatccagcttttatagtaactgatgatattggtataataagtagagaatatggacaatatcctggagttttagttgagattttaagaagagttaatacaagaaaacagaagggttgtgcactttcattaactgaggcttttggatcttga

SEQ ID NO: 10 - protein, 370 aa, Clostridium acetobutylicum
MTSYVDKQEITASSEIDDLIFSSDPLVWSYDEQEKIRKKLVLDAFRHHYKHCQEYRHYCQAHKVDDNITEIDDIPVFPTSVFKFTRLLTSNENEIESWFTSSGTNGLKSQVPRDRLSIERLLGSVSYGMKYIGSWFDHQMELVNLGPDRFNAHNIWFKYVMSLVELLYPTSFTVTEEHIDFVQTLNSLERIKHQGKDICLIGSPYFIYLLCRYMKDKNISFSGDKSLYIITGGGWKSYEKESLKRNDFNHLLFDTFNLSNINQIRDIFNQVELNTCFFEDEMQRKHVPPWVYARALDPETLKPVPDGMPGLMSYMDASSTSYPAFIVTDDIGIISREYGQYPGVLVEILRRVNTRKQKGCALSLTEAFGS

SEQ ID NO: 11 - DNA, 7803 bp, Artificial Sequence
  Description of Artificial Sequence Synthetic construct
aattcgaattctcagactcaaatagaacaggattctaaagacttaagagcagctgtagatcgtgattttagtacgatagagccaacattgagaaattatggggcaacggaagcacaacttgaagacgccagagccaaaatacacaagcttaaccaagaacagaggttatacaaatgacagttaatacagaggcactaataaacagcctaggcaagtcctaccaagaaatttttgatgaagggctaattccttataggaataagccaagtggttctcctggggtgcctaatatttgtattgacatggtgaaagaggggatttttttgtcgtttgaacggaatagtaaaatattaaacgaaattactttaagattgcttagagacgataaagctttgtttatatttccaaatgaattgccatcaccgttgaagcattctatggataggggatgggttagagaaaatttaggtgatctgattaaatcaataccaccgagacaaattttaaaaaggcagtttggttggaaagatctatatcgttttacggatgaaatcagtatgcagatttcttatgatttacgtgaacaggttaattcagtgactttcttgcttacatcagacgtgagttggtaatttaatatatatacccttcatccttcaagttgctgctttgttggctgctttctctcaccccagtcacatagttatctatgctcctggggattcgttcacttgccgccgcgctgcaacttgaaatctattgggtatatgctattggtaattatggaaaattgcctgatttatatataacttaacttgtaaaccagataataatttacatgaatattatcacgtataaaaaaattgcgattcttttaatttgaaatagttcaatttaattgaaactttttattaacaaatcttgttgatgtgaaaattttcgtttgctattttaacagatattgttaaacggagaaggcagcatgttgatgattcactcagccagactgacagttttaagcggaaaattgcagagtatgatcgcattctgataaaggttacaggtcactcgcaaccagaatttcatctttgtatattttgttttgttatttacgttgcagcaagacaaaaatagaagaaacaaatatttatacaacccgtttgcaagagggttaaacagcaatttaagttgaaattgccctattaaatggagcatgcggatcctcgactttttaacaaaatatattgataaaaataataggatccgggcccctcgagaggaggatggcaaatatgaataaaaagatatcatttattataaatggaagagttgaaatatttcctgagtcagatgatttagtacaatctataaattttggtgataattctgttcatcttccagtacttaatgattcacaggttaagaatattatagattataatgagaataatgagcttcagcttcataatattataaattttctttatacagtaggacagagatggaagaatgaggagtatagcagaagaagaacttatataagagatcttaagagatatatgggttatagtgaggaaatggcaaaattagaagctaattggatttcaatgatattatgttctaagggaggtttatatgatttagttaaaaatgaattaggaagtagacatattatggatgaatggttacctcaagatgaatcatatataagagcatttccaaaaggtaaaagtgtacatcttttaacaggaaatgttcctttaagtggagtactttcaattttaagagctatacttactaaaaatcagtgcattataaagacatctagtactgatccatttacagcaaatgctttagcacttagttttatagatgttgatcctcatcatccagtaactagatctttaagtgttgtatattggcaacatcaaggtgatatttcacttgctaaagaaataatgcaacatgcagatgttgtagttgcttggggaggtgaagatgcaattaattgggctgtaaagcacgcacctccagatatagatgttatgaaatttggacctaaaaagtctttttgtattatagataatccagtagatttagttagtgctgcaacaggtgctgcacatgatgtatgcttttatgatcagcaggcttgtttttcaactcaaaatatatattatatgggatcacattatgaagaatttaaacttgcattaattgaaaaacttaatttatatgctcatatacttccaaatacaaagaaagattttgatgaaaaggcagcttatagtttagttcagaaagaatgtttatttgcaggacttaaagtagaagttgatgtacatcaaagatggatggttattgaatcaaatgctggtgtagaattaaatcagccacttggaagatgcgtttatttacatcatgtagataatatagagcaaattttaccttatgttagaaagaataaaactcaaacaatatctgtatttccatgggaagcagctttaaagtatagagatcttttagcacttaaaggtgctgaaagaattgttgaggcaggaatgaataatatatttagagtaggtggtgctcatgatggaatgaggcctttacagagacttgttacttatataagtcatgaaagaccaagtcattatacagcaaaagatgtagctgtagagattgagcaaactagatttttagaagaagataagtttttagtatttgttccttaataggaggtaaaagaatatggaaaataaaagtagatataagacaatagatcatgttatttgtgtagaggagaatagaaagatacatgtttgggaaactttacctaaagaaaattcaccaaaaagaaaaaatacacttattatagcatctggatttgctagaagaatggatcattttgctggtttagctgaatatttatctcaaaatggatttcatgtaattagatatgattcattacatcatgttggtttaagttcaggaactatagatgaatttacaatgtcaattggtaagcagagtttacttgcagtagttgattggttaaatactagaaaaataaataatcttggaatgttagctagttcattatctgcaagaatagcttatgcaagtctttcagagattaatgtatcttttcttataacagctgttggtgtagttaatttaagatatactttagaaagagcacttggatttgattatcttagccttcctattgatgaattaccagataatcttgattttgagggacataagttaggtgctgaagtatttgcaagagattgctttgattcaggatgggaagatcttacatctactataaatagtatgatgcacttagatattccttttatagcttttacagcaaataatgatgattgggttaaacaagatgaggtaattactcttctttctagtataagaagtcatcagtgtaaaatatattcacttttaggttctagtcatgatcttggagaaaatttagttgtattaagaaatttttatcaatcagttacaaaggctgcaattgctatggataatggttgccttgatatagatgtagatattatagaaccatcttttgagcatttaactattgcagctgttaatgaaagaagaatgaaaatagaaatagagaatcaagtaattagtttaagttaaaacctataccaatagatttcgagttgcagcgcggcggcaagtgaacgcattcccaggagcatagataactctgtgactggggtgcgtgaaagcagccaacaaagcagcaacttgaaggatgaagggtatattgggatagatagttaactctatcactcaaatagaaatatactgcaggcggccgcaggaggactctctatgaaatttggaaattttttacttacatatcaacctccacagtttagtcaaactgaagttatgaagagattagtaaaacttggtagaatatcagaggaatgtggatttgatacagtttggttacttgaacatcattttactgagtttggtcttttaggaaatccttatgtagcagctgcatatttacttggtgctacaaagaaattaaatgtaggtacagcagctattgttttacctacagcacatcctgttagacagttagaagaagtaaatcttttagatcaaatgtctaaaggtagatttagatttggaatatgcagaggattatataataaggattttagagtttttggtactgatatgaataatagtagggctcttatggagtgttggtataaattaattagaaatggaatgacagaaggttatatggaagcagataatgagcatataaagtttcataaagtaaaagtacttccaactgcttattcacagggaggtgcacctatttatgtagttgctgaatctgcaagtacaactgaatgggctgcacagcatggattaccaatgatactttcatggattataaatacaaatgagaagaaagctcaaatagaattatataatgaagtagcacaagagtatggacatgatattcataatatagatcattgcctttcttatattactagtgttgatcatgattcaatgaaagctaaagaaatatgtagaaattttttaggtcattggtatgattcttatgtaaatgcaacaactatttttgatgatagtgataaaacaaagggatatgattttaataaaggtcagtggagagattttgttcttaaaggacataagaatactaatagaagagtagattattcatatgaaataaatcctgttggaactccacaagagtgtattgatataatacaaactgatattgatgctacaggaatatctaatatttgctgtggatttgaagcaaatggtactgtagatgaaataattagtagtatgaagttatttcagtctgatgttatgccttttcttaaggagaaacaaagaagtttactttattagctaaggaggaaaatgaaatgaaatttggattattttttcttaattttataaatagtacaactattcaagaacagtcaatagcaagaatgcaggagattacagagtatgttgataagctaaattttgagcagattcttgtatgtgaaaatcatttttcagataatggtgttgtaggtgctcctttaactgttagtggttttttattaggacttacagaaaaaattaagataggttcattaaatcatgtaattactacacatcatccagttagaatagcagaagaggcttgccttttagatcaactttctgaaggaagatttatattaggttttagtgattgtgaaagaaaagatgagatgcacttttttaatagacctgaacaatatcaacaacaactttttgaagagtgctatgatattataaatgacgcattaactacaggatattgtaatccaaatggagatttttataattttcctaaaatttcagtaaatccacatgcttatactcagaatggtcctagaaagtatgttacagcaacttcttgtcatgtagttgaatgggcagctaagaagggtataccattaatttttaaatgggatgatagtaatgaagtaaaacatgagtatgctaagagatatcaagcaatagctggtgaatatggagttgatcttgcagaaattgatcatcaattaatgatattagttaattattcagaggattctgaaaaagctaaggaagagacaagagcatttataagtgattatattttagctatgcaccctaatgaaaattttgaaaaaaaacttgaggaaataataactgaaaattcagttggtgattatatggagtgcacaactgctgcaaaacttgcaatggaaaaatgtggagctaaaggtattcttttatcttttgaaagtatgtcagattttacacatcagattaatgcaatagatatagtaaatgataatattaagaaatatcatatgtaatataccctatggatttcaaggtgcatcgcgacggcaagggagcgaatccccgggagcatatacccaatagatttcaagttgcagtgcggcggcaagtgaacgcatccccaggagcatagataactatgtgactggggtaagtgaacgcagccaacaaagcagcagcttgaaagatgaagggtatagataacgatgtgaccggggtgcgtgaacgcagccaacaaagaggcaacttgaaagataacgggtataaaagggtatagcagtcactctgccatatcctttaatattagctgccggctagcaggaggtaaaacaggtatgacatcttatgttgataaacaagaaataactgcaagttcagagattgatgatttaatatttagttcagatcctcttgtatggtcttatgatgaacaggaaaagattagaaaaaagttagttcttgatgcttttagacatcattataaacattgtcaagagtatagacattattgccaggcacataaagtagatgataatataacagaaattgatgatataccagtttttcctacttcagtatttaagtttacaagattacttacttcaaatgaaaatgagattgaatcatggtttacaagttcaggaactaatggtttaaaatctcaagttccaagagatagacttagtatagaaagacttttaggatcagtatcttatggtatgaagtatataggaagttggtttgatcatcaaatggagttagttaatcttggtcctgatagatttaatgctcataatatttggtttaaatatgtaatgtcacttgtagaacttttatatccaacaagttttactgtaacagaagagcatatagattttgttcagactttaaatagtcttgaaagaattaaacatcaaggaaaggatatatgtttaattggttcaccttattttatatatcttttatgcagatatatgaaagataagaatatttcttttagtggagataaatcactttatataataactggaggtggatggaaatcttatgaaaaggagagtttaaaaagaaatgattttaatcatcttttatttgatacttttaatctttcaaatattaatcaaataagagatatttttaatcaggtagaattaaatacatgtttttttgaggatgaaatgcaaagaaaacatgttccaccttgggtatatgcaagggctcttgatccagaaactttaaagcctgttccagatggtatgcctggacttatgtcttatatggatgcttcaagtactagttatccagcttttatagtaactgatgatattggtataataagtagagaatatggacaatatcctggagttttagttgagattttaagaagagttaatacaagaaaacagaagggttgtgcactttcattaactgaggcttttggatcttgaatgcatgtcgactctagagcatgctagtttctttggaaagaggagcagtcaaaggctcatttgttcaatgcttttgcgaaacgttttgtcgaactctaggcgaaggttctcgactttccccgcatcaggggtatatacaagtaaaaaagctcagggggtaaacctgagcttgggatgttgatttttaagtatgagatacatgggcggatttaaataacggagtcagtttggaaatatcaacggtcttttctgctttatcgaggctataagtttcttgcagttttaaccacaaccgcggagagctgccaagtacttgtgacagttttattgccatctctggcgtgactgctgctttacacgatactaaacgttgaaccgtagagggagcaacattcaatgcccgcgctaagttcacgaattc

(19) United States
(12) Patent Application Publication
(10) Pub. No.: US 2012/0264107 A1
(43) Pub. Date: Oct. 18, 2012

(54) REAL TIME MONITORING OF MICROBIAL ENZYMATIC PATHWAYS

(76) Inventor: Pamela Reilly Contag, San Jose, CA (US)

(21) Appl. No.: 13/353,233

(22) Filed: Jan. 18, 2012

Related U.S. Application Data
(63) Continuation of application No. 11/853,681, filed on Sep. 11, 2007, now abandoned.
(60) Provisional application No. 60/882,834, filed on Dec. 29, 2006.

(51) Int. Cl. C12Q 3/00; G06Q 90/00; C12N 15/52; C07C 31/12; C12N 1/21; C12M 1/36

(52) U.S. Cl. 435/3; 435/252.33; 435/252.3; 536/23.1; 435/286.1; 536/23.2; 568/840; 705/500

(57) ABSTRACT

This invention provides compositions and methods for monitoring and regulating the production of a target product of a biochemical pathway in an organism, such as butanol. A gene encoding a light-emitting reporter molecule, such as luciferase, is operatively linked with a transcription regulatory nucleotide sequence that regulates transcription of an enzyme in the pathway that signals the rate of production of the target product, such as butanol dehydrogenase. When a microorganism is transfected with such a reporter construct and cultured, the reporter is expressed contemporaneously with the enzyme. The amount of light produced by the reporter indicates amount of enzyme being produced which, in turn, signals the amount of target product being produced. When the reporter is measured in real time, it provides information that can be used to regulate culture conditions and to optimize production of the target product.

### CROSS-REFERENCE

This application is a Continuation Application of U.S. application Ser. No. 11/853,681, filed on Sep. 11, 2007, which claims the benefit of U.S. Provisional Application No. 60/882,834, filed Dec. 29, 2006, which are incorporated herein by reference in their entirety.

### BACKGROUND OF THE INVENTION

The flow of electrons along enzymatic pathways in a biological system is controlled by a number of factors. These factors include, for example, the concentration of substrates at various points in the pathways and positive and negative feedback by products of enzymatic transformation. In particular, certain target products may be toxic to a cell and thereby act as negative regulators of their own production. This is true, for example, for certain alcohols, such as ethanol and butanol.

Certain products of fermentative or synthetic pathways in an organism, such as alcohols, are commercially valuable. Such compounds, when produced by microorganisms, are produced in bulk quantities by culturing the microorganisms. However, the rate of production of desired target products changes over time, first increasing and then decreasing, as the cells move from exponential growth toward stasis and as the accumulation of toxic products inhibits their production.

It would be useful to maintain cultures in a state in which target production remained high over longer periods of time, thereby increasing the overall yield of commercially valuable products.

### SUMMARY OF THE INVENTION

In one aspect this invention provides a recombinant nucleic acid molecule comprising a transcription regulatory nucleotide sequence operatively linked with a nucleotide sequence encoding a self-contained light-emitting reporter, wherein the transcription regulatory nucleotide sequence regulates expression of a gene that signals production of a target product of a fermentative or synthetic pathway in a cell. In one embodiment of this invention, the transcription regulatory nucleotide sequence is a bacterial transcription regulatory nucleotide sequence, wherein the transcription regulatory nucleotide sequence regulates expression of a gene encoding an enzyme along the pathway and changes in expression of the reporter are positively correlated with changes in production of the target product. Alternatively, in another embodiment of this invention, changes in the expression of the reporter are negatively correlated with changes in production of the target product. In one embodiment of this invention, the expression of the reporter increases or decreases with increasing production of target product. In another embodiment of this invention, the expression of the reporter increases or decreases with decreasing production of target product.

In one embodiment of this invention, the target product is an end product. In a further embodiment of this invention the end product is acetone, ethanol, or butanol. In one embodiment of this invention, the target product is an acid intermediate. In a further embodiment of this invention the acid intermediate is acetate, butyrate, or lactate.

In one embodiment of this invention, the pathway is an anaerobic pathway. In another embodiment of this invention, the pathway is a fermentation pathway. In a further embodiment of this invention, the pathway is a substrate utilization pathway selected from gluconeogenesis, glycolysis, Entner-Doudoroff pathway or non-oxidative pentose phosphate pathway. In another embodiment of this invention, the bacterium converts hexoses, pentoses or amino acids into acids or alcohols.

In a one embodiment of this invention, the gene encodes an enzyme along a pathway leading from acetyl CoA to butanol or a branch of that pathway. In a further embodiment of this invention, the gene encodes butanol dehydrogenase, butyraldehyde dehydrogenase, ethanol dehydrogenase, acid aldehyde dehydrogenase, acetoacetate decarboxylase, butyrate kinase, phosphobutyryltransferase, phosphotransacetylase, acetate kinase, acyl CoA transferase, lactate dehydrogenase, or butyl CoA transferase. In another embodiment of this invention, the transcription regulatory nucleotide sequence is from Clostridium, E. coli, Z. mobilis, or S. cerevisiae.

In one embodiment of this invention, the self-contained light-emitting reporter is luminescent. In a further embodiment of this invention, the luminescent reporter comprises luciferase. In a still further embodiment of this invention, the luciferase is from Coleoptera, Photorhabdus, Vibrio, Gaussia, Diptera, Renilla. In another embodiment of this invention, the self-contained light-emitting reporter comprises a fluorescent reporter. In a further embodiment of this invention, the fluorescent reporter comprises green fluorescent protein (“GFP”). In another embodiment of this invention, the self-contained light-emitting reporter comprises a phosphorescent reporter.

In one aspect this invention provides a cell comprising a self-contained reporter construct that indicates when a synthetic or fermentative pathway has been induced or inhibited so as to affect the concentration of a target product of the pathway.

In another aspect this invention provides a cell comprising a recombinant nucleic acid molecule comprising a transcription regulatory nucleotide sequence operatively linked with a nucleotide sequence encoding a self-contained light-emitting reporter, wherein the transcription regulatory nucleotide sequence regulates expression of a gene that signals production of a target product of a fermentative or synthetic pathway in the cell. In one embodiment of this invention, the cell is a bacterial cell. In a further embodiment of this invention, the cell is Clostridium, E. coli, Z. mobilis, or S. cerevisiae. In one embodiment of this invention, the target product of the pathway in the cell is an end product. In a further embodiment of this invention, the end product of the pathway in the cell is butanol. In one embodiment of this invention, the gene encodes butanol dehydrogenase, butyraldehyde dehydrogenase, ethanol dehydrogenase, acid aldehyde dehydrogenase, acetoacetate decarboxylase, butyrate kinase, phosphobutyryltransferase, phosphotransacetylase, acetate kinase, acyl CoA transferase, lactate dehydrogenase, or butyl CoA transferase. In another embodiment of this invention, the cell contains one gene comprising a transcription regulatory nucleotide sequence operatively linked with a nucleotide sequence encoding a self-contained light-emitting reporter, wherein the transcription regulatory nucleotide sequence regulates expression of butyraldehyde dehydrogenase and additionally contains another gene comprising a transcription regulatory nucleotide sequence operatively linked with a nucleotide sequence encoding a self-contained light-emitting reporter, wherein the transcription regulatory nucleotide sequence regulates expression of butanol dehydrogenase.

In one aspect this invention provides a culture comprising cells that produce a target product of a synthetic or fermentative pathway in commercially valuable quantities and a light emitting reporter.

In another aspect this invention provides a method comprising: (a) culturing cells that comprise a recombinant nucleic acid molecule comprising a transcription regulatory nucleotide sequence operatively linked to a nucleotide sequence encoding a light-emitting reporter, wherein the transcription regulatory nucleotide sequence regulates expression of a gene that signals the production of a target product of a fermentative or synthetic pathway in the cell, whereby emission of light by the reporter signals production of the target product; (b) measuring the light emitted from the reporter in the culture; and (c) changing culture conditions to adjust production of the target product based on the production signaled by the emitted light.

In one embodiment of this invention, the light-emitting reporter is self-contained. In another embodiment of this invention, the target product is an end product. In a further embodiment of this invention, the target product is an acid intermediate. In one embodiment of this invention, the measuring of emitted light is performed in real time. In another embodiment of this invention, the emitted light increases or decreases with increasing production of target product. In a further embodiment of this invention, the emitted light increases or decreases with decreasing production of target product. In one embodiment of this invention, the cells are cultured in a culture container comprising a window and the light is measured through the window. In a further embodiment of this invention, the cells are cultured in a culture container comprising at least one light sensor within the culture that can sense the emitted light and directly or remotely signal a detector. In one embodiment of this invention, the cells are cultured in a culture container comprising a device that continuously flows culture fluid over a light sensor that senses the emitted light in the flow. In a further embodiment of this invention, if the target production decreases, culture conditions are changed to revive production, such actions comprise removal of the target product, adding nutrients, diluting the culture, or removing cells.

In one aspect this invention provides a method comprising: (a) culturing a recombinant cell under culture conditions to produce a target product, wherein the cell comprises a reporter construct that produces a light-based signal, the intensity of which indicates the level of production of the target product; (b) monitoring continuously over time the intensity of the signal in the culture at a plurality of different times to indicate the level of production of the target product at those times; and (c) altering the culture conditions in response to changes in target product production to set target product production to a desired level.

In another aspect this invention provides a culture that is monitored and controlled by software comprising: (a) code that receives information about the state of a cell or a cell culture; (b) code that determines whether and how culture conditions should be changed to optimize target production; (c) and code that transmits instructions on changing the culture conditions. In one embodiment of this invention, the code determines the state of the cell or cell culture.

In one aspect this invention provides a system comprising: (a) a container for culturing cells; (b) a photon detector for detecting light in a cell culture in the container; and (c) a computer controlled apparatus changes culture conditions in response to light detected by the detector. In one embodiment of this invention, the system further comprises a device that converts photons to electrons and electrons to photons. In an additional embodiment of this invention, the system further comprises a fermentation chamber comprising at least one window, or at least one light sensor within the culture that can directly or remotely signal a detector, or comprising sampling the culture, a continuous flow detector, whereby the culture fluid is passed over a detector/sensor that measures light. In one embodiment of this invention, the system further comprises a computer controlled apparatus that removes a target product from the container in response to signal from the computer indicating an amount of production of the target product.

In another aspect this invention provides a composition comprising substantially of butanol, and containing trace components from amaranth, or sweet sorghum, or both, and substantially free of petroleum by-products.

In one aspect this invention provides a business method comprising creating a joint venture between at least a first company that produces bioengineered cells that make a biofuel and a second company engaged in oil refining; running the joint venture wherein the first company provides a license to proprietary bioengineered bacterial strains that produce a biofuel, the second company sponsors research and development at the joint venture directed to biofuel production, and the second company purchases biofuel produced by the joint venture.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a number of biochemical pathways in Clostridium acetobutylicum that are active during the acidogenic or solventogenic phases. Enzymes that catalyze specific reactions are identified by letters as follows: (A) glyceraldehyde 3-phosphate dehydrogenase; (B) pyruvate-ferredoxin oxidoreductase; (C) NADH-ferredoxin oxidoreductase; (D) NADPH-ferredoxin oxidoreductase; (E) NADH rubredoxin oxidoreductase; (F) hydrogenase; (G) phosphotransacetylase (phosphate acetyltransferase), (pta, CAC1742); (H) acetate kinase (askA, CAC1743); (I) acetyl-CoA acetyltransferase (thiolase), (thil, CAP0078, and CAC2873)); (J) 3-hydroxybutyryl-CoA dehydrogenase; (K) crotonase (3-hydroxybutyryl-CoA dehydratase, beta-hydroxybutyryl-CoA dehydrogenase), (bad, CAC2708); (L) butyryl-CoA dehydrogenase (bcd, CAC2711); (M) phosphotransbutyrylase (phosphate butyltransferase) (ptb, CAC3076); (N) butyrate kinase, (buk, CAC3075, and CAC1660); (O) acetaldehyde dehydrogenase (possibly adhe1, CAP0162 and adhe, CAP0035); (P) ethanol dehydrogenase (adhe1, CAP0162; bdhB, CAC3298; and bdhA, CAC3299); (Q) butyraldehyde dehydrogenase, (adhe1, CAP0162 and adhe, CAP0035); (R) butanol dehydrogenase (adhe1, CAP0162; adhe, CAP0035; adh, CAP0059; bdhB, CAC3298; bdhA, CAC3299; and CAC3392); (S) butyrate-acetoacetate CoA-transferase (acetoacetyl-CoA:acetate/butyrate:CoA transferase), (ctfa, CAP0163(A) and ctfb, CAP0164(B); (T) acetoacetate decarboxylase (adc, CAP0165); (U) pyruvate decarboxylase (pdc, CAP0025). Select enzymes are further detailed in Table 1. Others can be found in readily available reference materials, such as on The Institute for Genomic Research's website (www.tigr.org).

### DETAILED DESCRIPTION OF THE INVENTION

### 1. Introduction

This invention provides methods and materials for increasing the total yield of commercially valuable products from organisms, in particular the yield from a culture of microorganisms. The methods are achieved by providing the organisms with a reporter system that indicates, in real time, the status of the biochemical pathway leading to the production of the desired product. The practitioner uses this information to alter culture conditions, using real time information, to “poise” the pathway in a desired state of target production. This can involve both increasing the rate of production and maintaining it over time. Thus, for example, if the reporter system indicates that the rate of product production is decreasing, the practitioner can modify culture conditions to increase production by, for example, adding substrate or nutrients, diluting the culture, removing cells, removing toxic products or changing environmental conditions such as agitation rate, atmospheric pressure, or temperature. This process can be performed by a computer-run system that includes computer code that receives and processes information about the status of a culture, executes an algorithm that determines whether and how culture conditions need to be changed to change the rate of production of the target and sends instructions to an apparatus; and an apparatus that executes the instructions to alter the culture conditions.

The state of a biochemical pathway is reflected by the level of production of enzymes that catalyze reactions of substrates toward or away from production of the target. One can obtain useful information both from the absolute rate of enzyme production and changes in that rate. For example, a high level of production of an enzyme that catalyzes the transformation of a precursor into a target indicates that product is being produced at a high level. Increasing levels of production of the enzyme over time also indicate that production of the target is increasing. Conversely, low levels of enzyme production or decreasing rates of enzyme production indicate low levels or decreasing rate of target production, respectively. On the other hand, high rates or increasing rates of production of an enzyme that diverts a substrate away from the production of a target indicate that production of the target is low or decreasing. Sub-optimal levels of production provide cause for intervening in the process to alter conditions to those that favor increased production of the target.

The reporter constructs of this invention provide means to measure the level of production of signal enzymes without the need to measure enzyme activity directly. In these constructs a transcription regulatory nucleotide sequence that regulates the expression of a signal enzyme in the system is coupled to a reporter gene so that the regulatory sequence regulates expression of the reporter gene. Thus, the expression level of the reporter mirrors the expression level of the signal enzyme in the system.

One aspect of the invention is controlling culture conditions to poise a culture to maintain pathways at desired levels of output. This involves, in part, measuring promoter activity while it is in progress and reporting the measurements quickly enough to allow the culture conditions to be acted upon to regulate pathway activity before culture conditions have significantly changed. Thus, monitoring and regulation of culture conditions occurs in real time. The reporter gene is selected to produce a reporter signal that can be measured in real time. A particularly useful class of reporters for this purpose is the class that emits light. In particular, this invention contemplates the luminescent protein, luciferase. Light can easily be measured electronically and electronic signals can be easily read.

This invention contemplates the use of these methods to monitor the production of any product of a synthetic or fermentative pathway. However, the method finds particular use in the production by microorganisms of solvents useful as fuels. In particular, this invention contemplates using the methods of the invention for regulating the production of butanol, a high value biofuel, in C. acetobutylicum, C. beijerinckii, C. puniceum, or C. saccharobutylicum.

### 2. Enzymatic Pathways Producing Targets of Interest

2.1. Pathways, Products and Signaling Enzymes

This invention is useful for monitoring and regulating the production of compounds of interest by a biochemical pathway, typically, but not exclusively, in vivo. A biochemical pathway is a sequence of enzymatic or other reactions by which one biological compound is converted to another. This invention contemplates, in particular, monitoring and regulating fermentative or synthetic biochemical pathways. This invention can be employed in both prokaryotic and eukaryotic systems. A biochemical pathway “target product” is a compound produced by an organism or an in vitro system wherein the product is the desired compound to be produced from the pathway. The target product can be a pathway “end product.” A pathway end product is a compound produced by an organism or an in vitro system wherein no further conversion of the compound is possible because there is no enzyme available that converts the compound to another compound. For example, no further enzymatic conversion is possible in a microorganism because, there is no gene in the genome that encodes such an enzyme. Examples of end products in Clostridia include the solvents: acetone, butanol and ethanol.

A target product can also be a biochemical pathway intermediate wherein further conversion of the compound is possible. In Clostridia, pathway intermediates include “acid intermediates.” The acid intermediates, acetate and butyrate, accumulate in the culture media when Clostridia is in the acidogenic culture phase. Later in the solventogenic phase, these acid intermediates will be reassimilated and used to synthesize solvents. Another acid intermediate, lactate, accumulates in the culture media when Clostridia is cultured under conditions of iron limitation and high pH.

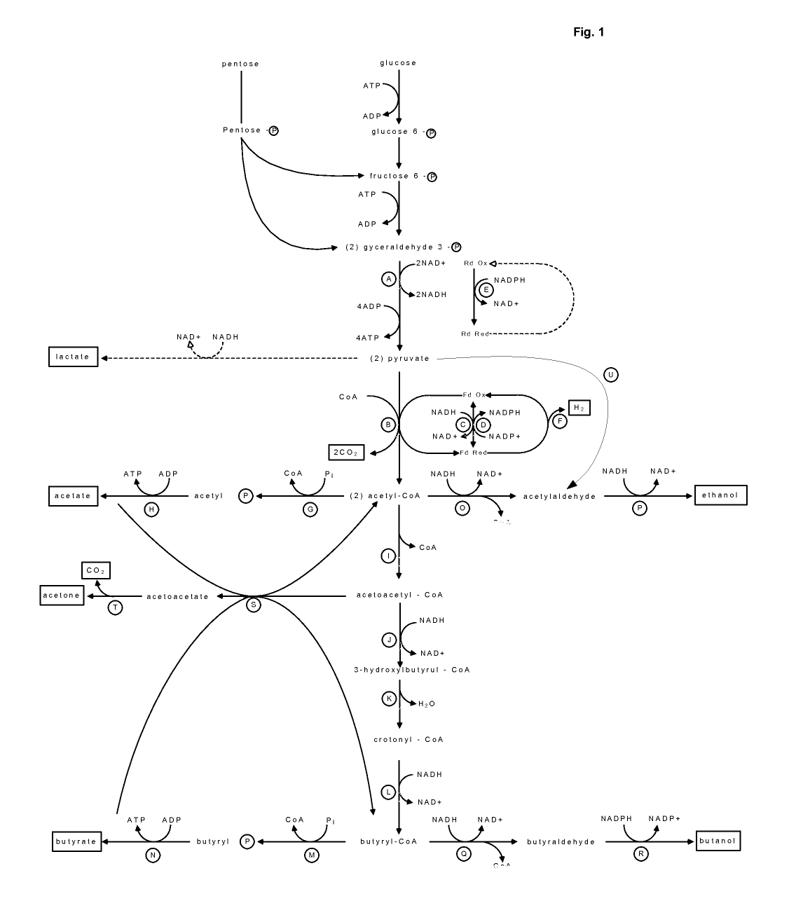

Enzymes whose expression provides information about the production of a target product in a system are said to “signal” production of the product and are also referred to herein as “signal enzymes.” With target products that are pathway end products, any enzyme that converts an intermediate of the pathway into another intermediate or into the end product itself, can be a signal enzyme. In general, enzymes that are the last enzyme in a pathway are better signal enzymes for the production of end products than those enzymes that are further up the pathway. For example, in C. acetobutylicum, the dehydrogenases that catalyze the reduction of butyraldehyde to butanol (Step R, FIG. 1) represent useful signal enzymes in that their expression directly indicates the rate of butanol production. Accordingly, a decrease in signal from a reporter operatively linked to this promoter indicates that culture conditions should be changed to increase the rate of butanol production.

In pathways where there is little to no diversion of the intermediate that is transformed in the last reaction to generate the end product, the enzymes that catalyze the production of the last intermediates in the pathways (two steps away from the end product) also function as excellent signal enzymes. For example, when C. acetobutylicum is in the solventogenic phase, butyraldehyde dehydrogenase (Step Q, FIG. 1) will function as an ideal signal enzyme since all the butyraldehyde produced by the enzyme will subsequently be converted to butanol. Therefore, the rate of butyraldehyde dehydrogenase synthesis will directly signal the rate of butanol production.

Similarly, where the target products are intermediates in biochemical pathways, the enzymes that catalyze the production of the intermediates are also excellent signal enzymes. For example, in C. acetobutylicum acetate kinase or butyrate kinase make ideal signal enzymes in that their rate of synthesis will indicate the rate of production of the acid intermediates acetate and butyrate, respectively. (Steps H and N, FIG. 1.) Where there is no diversion of the intermediates used to make the target intermediates, the enzymes that catalyze these reactions (two steps up the biochemical pathway) are also excellent signal enzymes. For example, in C. acetobutylicum phosphotransacetylase and phosphotransbutyrylase will make excellent signal enzymes for monitoring the production of acetate and butyrate, respectively. (Steps G and M, FIG. 1.)

Additionally, enzymes that recycle intermediates, such that these compounds become available to the fermentative or synthetic pathway of interest are also signal enzymes. For example, in C. acetobutylicum, the acetoacetyl-CoA:acetate/butyrate:CoA transferase complex recycles acetate and butyrate into acetyl-CoA and butyryl-CoA, respectively. (Step S, FIG. 1.) The use of either subunit of the acetoacetyl-CoA:acetate/butyrate:CoA transferase complex as a signal enzyme would indicate the rate of recycling of the acid intermediates. The appearance of the signal would also indicate the shift from the acidiogenic phase wherein the acid intermediates accumulate, to the solventogenic phase of culture wherein the acid intermediates are reassimilated by the microorganisms and then converted to solvents. Accordingly, an increase in signal from such an enzyme would indicate that culture conditions need not be altered for continued production of the target.

Conversely, enzymes that divert intermediates away from target pathways can also be used as signal enzymes, since the appearance of a signal and any subsequent increase in signal strength indicates that the rate of the production of the target product is decreasing thereby indicating that corrective action may need to be taken. For example, in C. acetobutylicum, if acid intermediates are the desired target, the appearance of a signal from butyraldehyde dehydrogenase (Step Q, FIG. 1) would indicate that the culture is shifting to the solventogenic phase whereby the accumulation of acid intermediates cease and actually decrease as they are reassimilated for solvent production.

2.2. Use of Branch Point Enzymes as Signaling Enzymes

The use of enzymes that occupy a position on the fermentative pathway immediately above or below where a branch point occurs that draws substrate away from a pathway would not be as informative to the status of the culture as would an enzyme further along the desired fermentative pathway, unless the organism had been engineered to either negate or down regulate the expression of an enzyme on the competing pathway. For example, in C. acetobutylicum, the use of acetyl-CoA acetyltransferase (Step I, FIG. 1) would be more informative of butanol production if the gene encoding an enzyme on a competing pathway such as acetaldehyde dehydrogenase is down regulated or deleted, thereby allowing more acetyl-CoA to be available for butanol production instead of ethanol production.

2.3. Use of Signaling Enzymes to Measure Viability of Culture

Reporters can be placed higher up in a metabolic pathway, that while not signaling for the production of a particular product can be used to provide information regarding the overall status of the culture in terms of carbon and electron flow and hence, organismic health. For example, in C. acetobutylicum, the use of glyceraldehyde-3-phosphate dehydrogenase (Step A, FIG. 1) as a signal enzyme would not provide as concise information on butanol production as would the use of an enzyme further down the butylic pathway such as butyraldehyde dehydrogenase (Step Q, FIG. 1). However, the use of an enzyme like glyceraldehyde-3-phosphate dehydrogenase would signal the overall metabolic rate of the culture which could then be used as a way to control the feed rate of media to the culture. Similarly, thiolase (acetyl coenzyme A acetyltransferase; Step I, FIG. 1) could also be used to provide information regarding the overall status of the culture.

2.4 Fermentative Pathways

A fermentative pathway is a metabolic pathway that proceeds anaerobically, wherein an organic molecule functions as the terminal electron acceptor rather than oxygen, as happens with oxidative phosphorylation under aerobic conditions. Glycolysis is an example of a wide-spread fermentative pathway in bacteria (C. acetobylicium and E. coli) and yeast. During glycolysis, cells convert simple sugars, such as glucose, into pyruvate with a net production of ATP and NADH. At least 95% of the pyruvate is consumed in short pathways which regenerate NAD⁺, an obligate requirement for continued glycolysis and ATP production. The waste or end products of these NAD⁺ regeneration systems are referred to as fermentation products. Depending upon the organism and culturing conditions, pyruvate is ultimately converted into end products such as organic acids (formate, acetate, lactate, pyruvate, butyrate, succinic, dicarboxylic acids, adipic acid, and amino acids), and neutral solvents (ethanol, butanol, acetone, 1,3-propanediol, 2,3-propanediol, acetaldehyde, butyraldehyde, 2,3-butanediol).

The Comprehensive Microbial Resource (CMR) of TIGR lists nine types of fermentation pathways in its atlas based on the fermentative end product: homolactic acid (lactic acid); heterolactic acid (lactic acid), ethanolic, propionic acid, mixed (formic and acetic acid), butanediol, butyric acid, amino acid, and methanogenesis. The method of this invention can be used in any of the fermentative pathways described above. The fermentative pathways described in this invention can be naturally occurring or engineered.

Solvents are a class of end products produced by microbes that have special commercial value. These include, for example, alcohols (ethanol, butanol, propanol, isopropanol, 1,3-propanediol, 2,3-propanediol, 2,3-butanediol, glycerol), ketones (acetone) and aldehydes (acetaldehyde, butyraldehyde). FIG. 1 illustrates the production of the solvents acetone, butanol and ethanol in C. acetobutylicum.

2.5 Solvent Production in Clostridia

The bacterium C. acetobutylicum was first identified by Weizmann during the period of 1912 to 1914 while he was searching for a fermentative source for butanol or isoamyl alcohol that could be used to make butadiene or isoprene and thereby supply the developing market for synthetic rubber. (Jones D. T., and Woods, D. R. Acetone-butanol fermentation revisited. Microbio. Rev. 50:484-524, 1986.) C. acetobutylicum co-produces the solvents acetone, butanol and ethanol (ABE) in a ratio roughly 3:6:1. Hydrogen and carbon dioxide are also produced during fermentation by C. acetobutylicum.

Different species of butanol-producing Clostridia are known and they are differentiated mainly by the type and ratio of the solvents they produce. C. beijerinckii (synonym C. butylicum) produces solvents in approximately the same ratio as C. acetobutylicum and in some strains of C. beijerinckii isopropanol is produced in place of acetone. (George, H. A., et al. Acetone, isopropanol, and butanol production by Clostridium beijernickii (syn. Clostridium butylicum) and Clostridium. Aurantibutyricum. Appl. Environ. Microbiol. 45:1160-1163, 1983.) C. saccharobutylicum is the proposed name for a Clostridium species identified through genetic and physiologic traits from saccharolytic industrial strains. (Keis, S., et al. Emended descriptions of Clostridium acetobutylicum, and Clostridium beijerinckii and descriptions of Clostridium saccharoperbutylacetonicum sp. nov. and Clostridium saccharobutylicum sp. nov. Intl. J. System. Evol. Microbio. 51:2095-2103, 2001.) C. aurantibutyricum produces both acetone and isopropanol in addition to butanol. (George, H. A., supra.) C. tetanomorphum produces almost equimolar amounts of butanol and ethanol, but not other solvents. (Gottwald, M., et al. Formation of n-butanol from D-glucose by strains of “Clostridium tetanomorphum” group. Appl. Environ. Microbio. 48:573-576, 1984.)

Solvent production in batch cultures of C. acetobutylicum proceeds through two phases. In the first, termed the acidogenic phase, that occurs during the exponential growth phase, C. acetobutylicum produces hydrogen, carbon dioxide, acetate and butyrate. The accumulation of acids in the culture media lowers the pH. The transition to the second or solventogenic phase, occurs when the undissociated concentration of butyric acid in the culture reaches approximately 9 mM. (Hüsemann, M. H. W., and E. T. Papoutsakis. Solventogenesis in Clostridium acetobutylicum fermentations related to carboxylic acid and proton concentrations. Biotechnol. Bioeng. 32:843-852, 1988.) This phase begins when C. acetobutylicium reaches early stationary phase. (Davies, R. and Stephenson M. Studies on the acetone-butyl alcohol fermentation. I. Nutritional and other factors involved in the preparation of active suspensions of Clostridium acetobutylicum. Biochem. J. 35:1320-1331, 1941.) Here, acetone, butanol and ethanol are synthesized concomitantly from the reassimilated acids and the continued consumption of carbohydrates, raising the culture's pH. Hydrogen and carbon dioxide production continues.

When C. acetobutylicum is grown in batch culture different proportions of acids and solvents may be produced depending on the dilution rate and the medium composition. (U.S. Pat. No. 5,063,156.) The addition of acetate or propionate does not affect the initiation of solventogenesis, but will increase the total concentration of solvents produced. (Hüsemann, M. H. W., and E. T. Papoutsakis. Solventogenesis in Clostridium acetobutylicum fermentations related to carboxylic acid and proton concentrations. Biotechnol. Bioeng. 32:843-852, 1988.)

Solvent yields can also be changed by sparging the culture with CO gas. This causes a reversal of the butyrate production pathway with the resultant uptake of butyrate that is then unavailable as a subsequent substrate for acetone production. (Hartmanis, M. G. N., et al. Uptake and activation of acetate and butyrate in Clostridium acetobutylicum. Appl. Microbiol. Biotechnol. 20:66-71, 1984.)

Changing the fermentation temperature can also affect butanol and solvent yield. In batch fermentation experiments conducted with three different solvent-producing strains, solvent yields remained fairly constant at around 31% at 30° C. and 33° C., but decreased to 23-25% at 37° C. (McCutchan, W. N., and Hickey, R. J. The butanol-acetone fermentations. Ind. Fement. 1:347-388, 1954.) Similar results were obtained in a more recent study with C. acetobutylicum NCIB 852 in which solvent yields were found to decrease from 29% at 25° C. to 24% at 40° C., although the fermentation time decreased as the temperature was increased. (McNeil, B., and Kristiansen, B., Effect of temperature upon growth rate and solvent production in batch cultures of Clostridium acetobutylicum. Biotech Lett. 7:499-502, 1985.) The decrease in solvent yield appeared to reflect a decrease in acetone production, while the yield of butanol was unaffected.

In continuous culture, C. acetobutylicum can be maintained in three different stable metabolic states. Acidogenic, when grown at neutral pH on glucose, solventogenic when grown at low pH on glucose and alcohologenic when grown at neutral pH under conditions of high NAD(P)H availability. (Girbal, L. et al. Regulation of metabolic shifts in Clostridium acetobutylicum ATCC824, FEMS Microbiol. Rev. 17:287-297, 1995.) An acidogenic culture will switch to the solventogenic phase with a lowering of pH, a lowering of acetate and/or butyrate concentration, with growth limiting quantities of phosphate or sulfate, but plentiful nitrogen and carbon sources. (Bahl, H. Andersch, W, and Gottschalk G. Continuous production of acetone and butanol by Clostridium acetobutylicum in a two-stage phosphate limited chemostat. Eur. J. Appl. Microbiol. Biotechnol. 15:201-205, 1982; Bahl, H., and Gottschalk G., Parameters affecting solvent production by Clostridium acetobutylicum in continuous culture, p. 215-223. In Wang D. I. C. and Scott. C. D. (ed.), Biotechnology and bioengineering Symposium no. 14, Sixth Symposium on Biotechnology for Fuels and Chemicals, John Wiley & Sons, Inc., New York, 1984.)

The physiologic signals for solventogenesis induce the biosynthesis of all terminal enzymes that catalyze solvent production with a concomitantly decrease in acidogenic enzymatic activity. (Andersch, W., Hubert, B., and Gottschalk, G. Level of enzymes involved in acetate, butyrate, acetone and butanol formation by Clostridium acetobutylicum. Eur. J. Appl. Microbiol. Biotechnol. 18:327-332, 1983. Rogers, P. Genetics and biochemistry of Clostridium relevant to development of fermentation processes. Adv. Appl. Microbiol. 31:1-60, 1986.)

2.6 C. acetobutylicum as a Model for Solventogenic Selection and Engineering

C. acetobutylicum is amenable to conventional mutational methodologies such as the use of alkylating agents like ethylmethylsulfonate (EMS), N-methyl N′-nitro N-nitrosoguanidine (NG), ICR 191, nitrous acid, nitroquinoline-N-oxide, and triethylene melamine, and selection by growth on increasing concentrations of butanol, resistance to allyl alcohol, or for cellulase, xylanase or amylase activity. Through such strategies regulatory mutants have been identified, along with mutants with increased solvent production, greater tolerance for higher solvent concentrations, decreased production of acids, and greater amolytic activity. (U.S. Pat. No. 4,757,010; Rogers, P., and Palosaari, N. Clostridium acetobutylicum mutants that produce butyraldehyde and altered quantities of solvents. Appl. Env. Microbio. 53:2761-2766, 1987.)

Studies exploring the overexpression of homologous genes and the expression of heterologous genes in low G+C gram-positive organisms such as C. acetobutylicum have lagged those of higher G+C organisms like E. coli, because low G+C gram-positive organisms are genetically distinct based on codon usage, amino acid usage and base content. They therefore required the design of new vectors and the sequencing and use of appropriate regulatory sequences. (C. acetobutylicum has 29% GC content compared to E. coli with 50% GC content.) These have been achieved and the study and use of low G+C gram-positive organisms is proceeding apace. (Gram-positive/negative shuttle vectors, U.S. Pat. No. 6,737,245; transposons, U.S. Pat. No. 7,056,728; bacteriaphages, Reid S. J. et al. Transformation of Clostridium acetobutylicum Protoplasts with Bacteriophage DNA. Appl Environ Microbiol. 1983 January; 45(1):305-307.) Therefore, C. acetobutylicum is an attractive host organism for the methods of this invention.

2.7 Butanol Production in C. acetobutylicum

For the production of butanol by C. acetobutylicum, the most appropriate enzymes for monitoring of butanol productivity are bdhB, (CAC3298) an aldehyde-alcohol dehydrogenase (Step R, FIG. 1); CAC3392, a NADH-dependent butanol dehydrogenase (Step R, FIG. 1); adh, (CAP0059) an alcohol dehydrogenase (Step R, FIG. 1); and adhe1 (CAP0162) an alcohol dehydrogenase/acetaldehyde dehydrogenase (Step 10, FIG. 1). Their attributes are described more fully below in the section on positive signal enzymes.

2.7.1 Butylic (Butanol Production) Pathway

For butanol production, glucose is first converted by way of glycolysis to pyruvate. The enzyme, glyceraldehyde-3-phosphate dehydrogenase catalyzes the last enzymatic step, the conversion of glyceraldehyde-3-phosphate to pyruvate. (Step A, FIG. 1.) Next, pyruvate is converted to acetyl-CoA with the concomitant loss of a molecule of carbon dioxide by the enzyme pyruvate-ferredoxin oxidoreductase. (Step B, FIG. 1.) Two acetyl CoA molecules are then condensed to acetoacetyl-CoA by acetyl-CoA acetyltransferases (thil, (thiolase), CAP0078; and CAC2873) with the production of one free CoA group. (Step I, FIG. 1.) Acetoacetyl-CoA is converted to 3-hydroxybutyrl-CoA (β-hydroxybutyrl-CoA) by 3-hydroxybutyrl-CoA dehydrogenase (hbd, CAC2708) a process that requires the oxidation of NADH to NAD⁺. (Step J, FIG. 1.) 3-hydroxybutyrl-CoA is then converted to crotonyl-CoA by crotonase (crt, CAC2712) with the concomitant loss of a molecule of water. (Step K, FIG. 1.) Crotonyl-CoA is converted to butyryl-CoA by butyryl-CoA dehydrogenase (bcd, CAC2711) with the concomitant oxidation of NADH to NAD⁺. (Step L, FIG. 1.) Butyryl-CoA is reduced to butyraldehyde by butyraldehyde dehydrogenase (adhe, CAP0035, and adhe1, CAP0162) and NADH. (Step Q, FIG. 1.) Finally, butyraldehyde is reduced to butanol by dehydrogenases (adhe, CAP0035, adhe1, CAP0162, adh, CAP0059, bdhA, CAC3299, bdhB, CAC3298, and CAC3392) and NADPH. (Step R, FIG. 1.)

During the start of solventogenesis, butyrate and acetate are reassimilated by C. acetobutylicum and converted by the ctfa/ctfb complex (acetoacetyl-CoA:acetate/butyrate:CoA transferase) (Step S, FIG. 1) into butyryl-CoA and acetyl-CoA, respectively. These intermediates can then flow down to the butylic pathway. Butyrate production does not end with the initiation of solventogenesis, because the conversion of butyryl-phosphate to butyrate is one of the few mechanism available to C. acetobutylicum for the synthesis of ATP. (Step N, FIG. 1.) Butyrate produced during solventogenesis is recycled back to butyryl-CoA by the ctfa/ctfb complex (acetoacetyl-CoA:acetate/butyrate:CoA transferase). (Step S, FIG. 1.)

2.7.2 Signaling Enzymes to Provide Positive Feedback of Butanol Production

The onset of solventogenesis can be monitored by use of the transcription regulatory nucleotide sequence of the sol operon, found on the pSOL1 megaplasmid of C. acetobutylicum ATCC 824. The sol operon controls the transcription of three genes, adhE, CAP0035 (aldehyde-alcohol dehydrogenase), ctfA, CAP0163 (A), and ctfB, CAP0164(B) (butyrate-acetoacetate CoA-transferase subunits A and B) the expression of which increases approximately 10-fold with the initiation of solventogenesis. (Feustel, L., et al. Characterization and development of two reporter gene systems for Clostridium acetobutylicum. Appl. Environ. Microbiol. 70:798-803, 2004.) Also on the pSOL1 megaplasmid is adc, CAP0165, (acetoacetate decarboxylase) the transcription of which also increases approximately 10-fold with the onset of solventogenesis. (Feustel, L., et al. supra.)

The use of the transcription regulatory nucleotide sequence of the sol operon may be suboptimal for the monitoring of the later phase of solvent production since the gene product of adhE, butyraldehyde/butanol dehydrogenase, is active only during the onset of solventogenesis. During the later portion of solvent production another aldehyde-alcohol dehydrogenase, bdhB, found on its own monocistronic operon, takes over. (Petersen, D. J., et al. Molecular cloning of an alcohol (butanol) dehydrogenase gene cluster from Clostridium acetobutylicum ATCC 824. J. Bacteriol. 173:1831-1834, 1991; Sauer, U., and P. Dürre. Differential induction of genes related to solvent formation during the shift from acidogenesis to solventogenesis in continuous culture of Clostridium acetobutylicum. FEMS Microbiol. Lett. 125:115-120, 1995) The transcription regulatory nucleotide sequence of the bdhB operon, therefore, may be a more appropriate sequence to couple to a reporter gene especially since the aldehyde-alcohol dehydrogenase encoded for by bdhB is believed to be responsible for high butanol production. (Feustel, L., et al., supra.)

Other transcription regulatory nucleotide sequences of interest for monitoring butanol production include CAC3392 (NADH-dependent butanol dehydrogenase) and adh, CAP0059 (alcohol dehydrogenase), since these genes encode for enzymes used in the last step of butanol production, the reduction of butyraldehyde to butanol.

Additionally, the transcription regulatory nucleotide sequence for adhe1 (CAP0162, alcohol dehydrogenase/acetaldehyde dehydrogenase) could be used since butyraldehyde is one enzymatic step away from butanol and there are no recycling mechanisms for butyraldehyde.

bdhA, CAC3299 (NADH-dependent butanol dehydrogenase A), is however, an inappropriate choice for monitoring butanol production since it is expressed during the exponential growth phase and reaches a maximum as soon as the pH of the culture starts to drop. (Feustel et al., supra)

2.7.3 Use of Enzymes Above or Below a Branch Point as Signaling Enzymes

For butanol production in C. acetobutylicum, where enzymes further down a competing pathway have been deleted or down regulated, enzymes immediately above or below a branch point could be used as signaling enzymes. For example, if an enzyme in the acetone production pathway like acetoacetate decarboxylase is deleted (Step T, FIG. 1), then the enzyme immediately branch point above the branch point, acetyl-CoA acetyltransferase (Step I, FIG. 1), can be used to monitor butanol production. Similarly, the enzymes below this branch point, 3-hydroxybutyryl-CoA dehydrogenase, crotonase, and butyryl-CoA dehydrogenase (Steps J, K, L, FIG. 1) can also be used to monitor butanol production.

2.7.4 Signaling Enzymes to Provide Negative Feedback of Butanol Production

Enzymatic activity along the butyric pathway comprising phosphate butyryltransferase (ptb, CAC3076) and butyrate kinases (buk, CAC1660 and buk, CAC3075) (Steps M and N, FIG. 1) signals the diversion of butyryl-CoA substrate away from the butylic pathway. The transcription regulatory nucleotide sequence of one of these enzymes can be coupled to a reporter gene to indicate that butanol production may be decreasing. Given the need for continued ATP production during solvenogenesis via the butyric pathway, the use of these transcription regulatory nucleotide sequences may be suboptimal. Several other competing pathways can draw intermediates away from the butylic pathway and the genes coding for the respective enzymes may represent useful transcription regulatory nucleotide sequences for the monitoring of butanol production. Lactate dehydrogenase can reduce pyruvate using lactate dehydrogenase into lactate. (Step U, FIG. 1.) No monitoring of pyruvate diversion is probably necessary, since lactate production in C. acetobutylicum is minimal except under conditions of iron limitation and high pH. (Bahl, H., et al. Nutritional factor affecting the ratio of solvents produced by Clostridium acetobutylicum. Appl. Environ. Microbiol. 52:169-172, 1986.) Pyruvate decarboxylase can convert pyruvate into acetaldehyde. (Step U, FIG. 1.) Acetyl-CoA can be drawn off to make acetate. (Steps G and H, FIG. 1.) Acetyl-CoA can also be drawn off to make ethanol. (Steps 0 and P, FIG. 1.) Acetoacetyl-CoA can be converted to acetone by way of acetoacetyl-CoA:acetate/butyrate-CoA transferase and acetoacetate decarboxylase. (Steps S and T, FIG. 1.)

2.7.5 Use of Multiple Signaling Enzymes

In the batch culture of C. acetobutylicum for the production of butanol, several constructs using luciferases with different spectral emissions can be incorporated into the various pathways to indicate the progress of the fermentation. A construct using the regulatory sequence of phosphotransacetylase (pta, CAC1742, Step G, FIG. 1), acetate kinase (askA, CAC1743, Step H, FIG. 1), phosphate butyryltransferase (ptb, CAC3076, Step M, FIG. 1), or butyrate kinases (CAC1660 and buk, CAC3075, Step N, FIG. 1) will signal the initiation and vigor of the acidogenic phase of culture. The signal strength of this construct can then be used to poise the culture to achieve the desired acid concentrations and cell mass. A decrease in the signal strength for this construct coupled with the appearance of a signal for a construct that utilizes the transcription regulatory nucleotide sequence for an enzyme in the butylic pathway indicates that the transition to solventogenesis is occurring. The culture conditions can be adjusted, if desired, to either delay this transition or to facilitate it. Once the culture is placed into the solventogenic phase, the signal strength of the construct utilizing the butylic enzyme transcription regulatory nucleotide sequence can then be used to monitor and control this phase of the culture for maximum solvent production.

Alternatively, in the batch culture of C. acetobutylicum for the production of butanol, several constructs can be utilized that have the same luciferase. This is possible because the spectral emissions of luciferase are pH dependent with a red shift occurring in an acidic environment. (Feustel, L., et al. supra.) Therefore, with the use of a transcription regulatory nucleotide sequence from an enzyme like phosphotransbutyrylase (ptb, CAC3076, Step M, FIG. 1) where its transcription is almost completely repressed at the onset of solventogenesis, a luciferase signal will be seen at the start of the acidogenic phase. As the pH decreases the emission peak will shift from 560 mm at a pH of 6.8 to 617 nm at a pH of about 5. If the second construct uses the transcription regulatory nucleotide sequence for a gene like bdhB that is expressed after solventogenesis is initiated, then there should be a decrease in signal strength and a shift of the emission spectra as the luciferase produced by the ptb construct decays or becomes inactivated. This will then be followed by an increase in strength of the luciferase signal with a continued shift back to emissions peak seen at a more neutral pH with ongoing solventogenesis.

In the continuous culture of C. acetobutylicum for the production of butanol, several constructs using luciferases with different spectral emissions can be incorporated into the various pathways to indicate the status of the fermentation. The appearance of a signal from a construct that utilizes the regulatory sequence of phosphotransacetylase (pta, CAC1742, Step G FIG. 1), acetate kinase (askA, CAC1743, Step H, FIG. 1), phosphate butyryltransferase (ptb, CAC3076, Step M, FIG. 1) or butyrate kinases (buk, CAC1660 and buk, CAC3075, Step N, FIG. 1) will indicate that parameters of the culture are shifting away from those needed to maintain the culture in the solventogenic phase. Action can then be taken to adjust the culture conditions to return the culture to the solventogenic phase. Because of the continual need for ATP synthesis by way of butyrate kinase activity, use of the transcription regulatory nucleotide sequences from phosphate butyryltransferase (ptb) or the butyrate kinases may be suboptimal.

TABLE 1
Select C. acetobutylicum Enzymes Involved in Acidogenesis or Solventogenesis

| Letter | Gene ID | Name | Definition |
|---|---|---|---|
| G | CAC1742 | pta | Phosphotransacetylase [another source called it |
| | | | Phosphate acetyltransferase] |
| H | CAC1743 | askA | Acetate kinase |
| I | CAC2873 | | Acetyl-CoA acetyltransferase |
| I | CAP0078 | thil | Acetyl coenzyme A acetyltransferase [thiolase] |
| J | CAC2708 | hbd | Beta-hydroxybutyryl-CoA dehydrogenase [Also listed |
| | | Also listed | as 3-hydroxybutyryl-CoA dehydrogenase] |
| | | as Hdb | |
| K | CAC2712 | crt | Crotonase [3-hydroxybutyryl-CoA dehydratase] |
| L | CAC2711 | bcd | Butyryl-CoA dehydrogenase |
| M | CAC3076 | ptb | Phosphate butyryltransferase |
| N | CAC1660 | | Butyrate kinase |
| N | CAC3075 | buk | Butyrate kinase, BUK |
| O | CAP0162 | adhe1 | Alcohol dehydrogenase/acetaldehyde dehydrogenase |
| | | | [aldehyde dehydrogenase (NAD+)] |
| O | CAP0035 | adhe | Aldehyde-alcohol dehydrogenase [ADHE1] |
| P | CAP0162 | adhe1 | Alcohol dehydrogenase/acetaldehyde dehydrogenase |
| | | | [aldehyde dehydrogenase (NAD+)] |
| P | CAP0036 | | Uncharacterized, ortholog of YgaT gene of B. subtillis |
| P | CAC3298 | bdhB | NADH-dependent butanol dehydrogenase B [BDH II] |
| P | CAC3299 | bdhA | NADH-dependent butanol dehydrogenase A [BDH I] |
| P | CAP0059 | adh | Alcohol dehydrogenase |
| Q | CAP0162 | adhe1 | Alcohol dehydrogenase/acetaldehyde dehydrogenase |
| | | | [aldehyde dehydrogenase (NAD+)] |
| Q | CAP0035 | adhe | Aldehyde-alcohol dehydrogenase [ADHE1] |
| R | CAP0059 | adh | Alcohol dehydrogenase |
| R | CAC3298 | bdhB | NADH-dependent butanol dehydrogenase B [BDH II] |
| R | CAC3299 | bdhA | NADH-dependent butanol dehydrogenase A [BDH I] |
| R | CAC3392 | | NADH-dependent butanol dehydrogenase |
| R | CAP0162 | adhe1 | Alcohol dehydrogenase/acetaldehyde dehydrogenase |
| | | | [aldehyde dehydrogenase (NAD+)] |
| R | CAP0035 | adhe | Aldehyde-alcohol dehydrogenase [ADHE1] |
| S | CAP0163(A) | ctfa | Butyrate-acetoacetate CoA-transferase subunit A |
| S | CAP0164(B) | ctfb | Butyrate-acetoacetate CoA-transferase subunit B |
| T | CAP0165 | adc | Acetoacetate decarboxylase |
| U | CAP0025 | pdc | Pyruvate decarboxylase |

2.8 Butyric Pathway

For the production of butyrate by C. acetobutylicum, the most appropriate enzymes for monitoring butyrate productivity are acetate kinase (ackA, CAC1743, Step H, FIG. 1) and butyrate kinase (buk CAC1660 and buk CAC3075, Step N, FIG. 1.) Their attributes are described more fully below in the section on positive signal enzymes.

The butyrate production path way is as follows. Glucose is first converted by way of glycolysis to pyruvate. The enzyme, glyceraldehyde-3-phosphate dehydrogenase catalyzes the last enzymatic step, the conversion of glyceraldehyde-3-phosphate to pyruvate. (Step A, FIG. 1.) Next, pyruvate is converted to acetyl-CoA with the concomitant loss of a molecule of carbon dioxide by the enzyme pyruvate-ferredoxin oxidoreductase. (Step B, FIG. 1.) Two acetyl CoA molecules are then condensed to acetoacetyl-CoA by acetyl-CoA acetyltransferases (thil, (thiolase), CAP0078, and CAC2873, Step I, FIG. 1) with the production of one free CoA group. Acetoacetyl-CoA is converted to 3-hydroxybutyrl-CoA (β-hydroxybutyrl-CoA) by 3-hydroxybutyrl-CoA dehydrogenase (hbd, CAC2708, Step J, FIG. 1) a process that requires the oxidation of NADH to NAD⁺. 3-hydroxybutyrl-CoA is then converted to crotonyl-CoA by crotonase (crt, CAC2712, Step K, FIG. 1) with the concomitant loss of a molecule of water. Crotonyl-CoA is converted to butyryl-CoA by butyryl-CoA dehydrogenase (bcd, CAC2711, Step L, FIG. 1) with the concomitant oxidation of NADH to NAD⁺. Butyryl-CoA is phosphorylated by phosphotransbutyrylase (ptb, CAC3076, Step M, FIG. 1) to make butyrylphosphate. Finally, butyrylphosphate is converted to butyrate by butyrate kinase (CAC1660 and buk, CAC3075, Step N, FIG. 1) with the production of one molecule of ATP.

2.8.1 Signaling Enzymes to Provide Positive Feedback of Butyrate Production

During acidogenesis, the expression of the genes coding for the enzymes that are responsible for the catalyzing the final steps of acetate and butyrate production, acetate kinase (ack, Step H, FIG. 1), and butyrate kinase (buk, Step N, FIG. 1), respectively, is high. (Durre, P. et al. Transcriptional regulation of solventogenesis in Clostridium acetobutylicum. J. Mol. Microbiol. Biotechnol. 4:295-300, 2002.) Therefore, their transcription regulatory nucleotide sequences represent ideal choices for the construction of signal enzyme constructs. Furthermore, since the substrates for acetate kinase and butyrate kinase, acetyl-phosphate and butyryl-phosphate, respectively, do not serve as substrates for competing reactions, the transcription regulatory nucleotide sequences of the enzymes that make these intermediate compounds can also be used to monitor the status of acidogenesis, particularly since these enzymes, phosphotransacetylase (pta, Step G, FIG. 1), and phosphotransbutyrylase (ptb, Step M, FIG. 1) are also highly expressed during acidogenesis.

2.8.2 Signaling Enzymes to Provide Negative Feedback of Butyrate Production

Several competing pathways can draw intermediates away from the butyric pathway. Lactate dehydrogenase can reduce pyruvate into lactate using lactate dehydrogenase. (Step U, FIG. 1.) Pyruvate decarboxylase can convert pyruvate into acetaldehyde. (Step U, FIG. 1.) Thereby, drawing pyruvate off to form ethanol. Acetyl-CoA can be drawn off make acetate. (Steps G and H, FIG. 1.) Acetyl-CoA can also be drawn off to make ethanol. (Steps 0 and P, FIG. 1.) Acetoacetyl-CoA can be converted to acetone by way of acetoacetyl-CoA:acetate/butyrate-CoA transferase and acetoacetate decarboxylase. (Steps S and T, FIG. 1.) Butyryl-CoA, can also be shunted to the production of butanol. (Steps Q and R, FIG. 1.)

Additionally, butyrate can be recycled by acetoacetyl-CoA:acetate/butyrate-CoA transferase back into butyryl-CoA, and from there be shunted to the synthesis of butanol. (Step S, FIG. 1.)

The two most appropriate sources for transcription regulatory nucleotide sequences for use in signal enzyme constructs are the transcription regulatory nucleotide sequence for the genes for the enzymes acetoacetyl-CoA:acetate/butyrate-CoA transferase, and butyraldehyde dehydrogense. Acetoacetyl-CoA:acetate/butyrate-CoA transferase (Step S, FIG. 1), converts reassimilated butyrate into butyryl-CoA that can be subsequently shunted to the butylic pathway. Butyraldehyde dehydrogense (Step R, FIG. 1), is the first enzyme in the butylic pathway and reduces butyryl-CoA to butyraldehyde, the immediate precursor to butanol. An alternate source for a transcription regulatory nucleotide sequence is the transcription regulatory nucleotide sequence for the butyryl-CoA dehydrogenase (Step Q, FIG. 1), that reduces butyryl-CoA to butyraldehyde, a substrate one step removed from butanol that cannot be drawn off to a competing use or be recycled.

2.9 Ethanologenic Pathway

In another embodiment, the practitioner uses the methods of this invention to regulate the production of ethanol. For ethanol production, glucose is first converted by way of glycolysis to pyruvate. The enzyme, glyceraldehyde-3-phosphate dehydrogenase catalyzes the last enzymatic step, the conversion of glyceraldehyde-3-phosphate to pyruvate. (Step A in FIG. 1.) From here, pyruvate can flow through two separate ethanologenic pathways as Clostridia is one of the few genera of bacteria that possess pyruvate decarboxylate. In one pathway, pyruvate is converted to acetyl-CoA with the concomitant loss of a molecule of carbon dioxide by the enzyme pyruvate-ferredoxin oxidoreductase. (Step B, FIG. 1.) Acetyl-CoA is then converted to acetylaldehyde by acetaldehyde dehydrogenase (Step O, FIG. 1) and NADH. Finally, acetylaldehyde is reduced to ethanol by dehydrogenase (bdhB, CAC3298; bdhA, CAC3299; and possibly adhe1, CAP0162, and CAP0035, Step P, FIG. 1) and NADH. In the other pathway, pyruvate is decarboxylated by pyruvate decarboxylase (Step U, FIG. 1) to form acetylaldehyde, that is then reduced to ethanol by dehydrogenases (bdhB, CAC3298; bdhA, CAC3299; and possibly adhe1, CAP0162, and CAP0035, Step P, FIG. 1) and NADH.

2.9.1 Signaling Enzymes to Provide Positive Feedback of Ethanol Production

Ethanol production can be directly monitored by designing a construct with the transcription regulatory nucleotide sequence for a dehydrogenase coupled to the reporter gene. Even though these enzymes are the last enzymes in the ethonologenic pathway and there are no competing uses for the intermediate acetylaldehyde, this method may give a signal that is out of proportion of actual ethanol production since the dehydrogenase are also used in the butylic pathway to reduce butyraldehyde to butanol. Alternatively, a better gauge of ethanol production could be had by the simultaneous monitoring of pyruvate decarboxylase and acetaldehyde activity through the use of two constructs, each using their respective transcription regulatory nucleotide sequence.

2.9.2 Signaling Enzymes to Provide Negative Feedback of Ethanol Production

Several competing pathways can draw intermediates away from the ethanolic pathway. Lactate dehydrogenase can reduce pyruvate using lactate dehydrogenase into lactate. (Step U, FIG. 1.)

Acetyl-CoA can be drawn off to make acetate. (Steps G and H, FIG. 1.) Acetyl-CoA can also be converted to acetoacetyl-CoA by acetyl-CoA acetyltransferase. (Step I, FIG. 1.) From here acetyl-CoA can be converted into acetone (Steps S and T, FIG. 1), butyrate (Steps J, K, L, M, and N, FIG. 1) or butanol (Steps J, K, L, Q, and R, FIG. 1.) Signaling enzyme constructs can be designed that use the transcription regulatory nucleotide sequences for phosphotransacetylase (Step G, FIG. 1) and acetyl-CoA acetyltransferase (Step I, FIG. 1) to monitor the diversion of the substrate acetyl-CoA away from the ethanologenic pathway. No monitoring of pyruvate diversion is probably necessary, unless culture conditions are of iron limitation and high pH.

2.10 Acetone Pathway

In another embodiment, the practitioner uses the methods of this invention to regulate the production of acetone. For the production of acetone, glucose is first converted by way of glycolysis to pyruvate. The enzyme, glyceraldehyde-3-phosphate dehydrogenase catalyzes the last enzymatic step, the conversion of glyceraldehyde-3-phosphate to pyruvate. (Step A, FIG. 1.) Next, pyruvate is converted to acetyl-CoA with the concomitant loss of a molecule of carbon dioxide by the enzyme pyruvate-ferredoxin oxidoreductase. (Step B, FIG. 1.) Two acetyl CoA molecules are then condensed to acetoacetyl-CoA by acetyl-CoA acetyltransferases (thil, (thiolase), CAP0078, and CAC2873) with the production of one free CoA group. (Step I, FIG. 1.) Acetoacetyl-CoA is converted to acetoacetate by acetoacetyl-CoA:acetate/butyrate CoA transferase. (Step S, FIG. 1.) Acetoacetate is converted to acetone by acetoacetate decarboxylase with the production of one molecule of carbon dioxide. (Step T, FIG. 1.)

2.10.1 Signaling Enzymes to Provide Positive Feedback of Acetone Production

On the pSOL1 megaplasmid, resides adc, CAP0165, (acetoacetate decarboxylase, Step T, FIG. 1) which is transcribed from its own promoter in the opposite direction of that of the sol operon. Transcription of acetoacetate decarboxylase occurs at the onset of the solventogenic phase and the activity of the enzyme was found to be stable throughout the solventogenic phase. (Gerischer, U., and Dune, P. mRNA analysis of the adc gene region of Clostridium acetobutylicum during the shift to solventogenesis. J. Bact. 174:426-433, 1992) Additionally, acetoacetate decarboxylase is the last enzyme in the acetone pathway. Therefore the use of the transcription regulatory nucleotide sequence for adc is ideal for the monitoring of acetone production. For the batch culture production of acetone, the subunits of the enzyme acetoacetyl-CoA:acetate/butyrate:CoA transferase could be useful since it converts acetate, an end product produced during the acidogenic phase into acetyl-CoA where it can serve as a substrate for acetyl-CoA acetyltransferase to make acetoacetyl-CoA. Acetoacetyl-CoA:acetate/butyrate:CoA transferase can then convert acetoacetyl-CoA into acetoacetate, the last intermediate in the acetone synthetic pathway. (Steps S, I, S, T, FIG. 1.) Similarly, in continuous solventogenic culture acetoacetyl-CoA:acetate/butyrate:CoA transferase activity can provide information on the rate of acetoacetate production, and therefore, indirectly the rate of acetone production.

2.10.2 Signaling Enzymes to Provide Negative Feedback of Acetone Production

Several competing pathways can draw intermediates away from the acetone pathway. Lactate dehydrogenase can reduce pyruvate using lactate dehydrogenase into lactate. (Step U, FIG. 1.) This, however, is minimal except under conditions of iron limitation and high pH. Acetyl-CoA can be drawn off make acetate. (Steps G and H, FIG. 1.) Acetyl-CoA can also be drawn off to make ethanol. (Steps 0 and P, FIG. 1.) The substrate acetoacetyl-CoA that is converted by acetoacetate decarboxylase into acetone can also be converted by acetoacetyl-CoA:acetate/butyrate:CoA transferase into butyryl-CoA, an intermediary for the production of butyrate or butanol. (Steps J, K, L, then branch point to M and N or Q and R, respectively, FIG. 1.) The activity of enzymes further along the butyric/butylic pathway from acetoacetyl-CoA can be useful since this will provide information on the production of intermediates and targets that are unavailable for acetone production. (Steps J, K, L, M, N, Q, and R. FIG. 1.) Use of the enzymes before the branch point in the butyric/butylic pathway, 3-hydroxybutyryl-CoA, crotonase, and butyryl-CoA dehydrogenase (Steps J, K, and L, FIG. 1) will provide information regarding diversion of substrate at all times (acidogenic and solventogenic) as opposed to the enzymes past the branch point (Steps M, N, Q, and R, FIG. 1) that will provide information on regarding a particular fermentative phase of the culture. Therefore, the use of the transcription regulatory nucleotide sequences for 3-hydroxybutyryl-CoA, crotonase, and butyryl-CoA dehydrogenase are preferred. It should be remembered that butyrate can be recycled by acetoacetyl-CoA:acetate/butyrate:CoA transferase into acetoacetate, that can serve as a substrate for acetone production. Therefore, signal enzymes based on transcription regulatory nucleotide sequences for hydroxybutyryl-CoA, crotonase, and butyryl-CoA dehydrogenase, phosphotransbutyrylase and butyrate kinase may provide too high of a signal (Steps J, K, L, M, and N, FIG. 1).

2.11 Solventogenesis in Other Microorganisms

Ethanologenic organisms like Zymomonas mobilis and Saccharomyces cerevisiae ferment one molecule of glucose into two molecules of ethanol and two molecules of CO₂. Two enzymatic steps are required. First pyruvate decarboxylase cleaves pyruvate into acetaldehyde and carbon dioxide. Then alcohol dehydrogenase regenerates NAD+ by transferring hydrogen equivalents from NADH to acetaldehyde, thereby producing ethanol. Z. mobilis is a bacterium commonly found in plant saps and honey relies on the Entner-Doudoroff pathway as a fermentative path. This shorter pathway yields only one ATP per glucose molecule. Z. mobilis possesses two alcohol dehydrogenase isozymes that catalyze the reduction of acetaldehyde to ethanol during fermentation, accompanied by the oxidation of NADH to NAD+.

The production of ethanol by S. cerevisiae is well known and results in the net production of two molecules of ATP for every molecule of glucose. Both Z. mobilis and S. cerevisiae have served as the source of heterologous genes for the production of ethanol in other microorganisms.

2.11.1 Solventogenesis in E. coli

The bacterium E. coli does not naturally possess the enzyme pyruvate decarboxylase and therefore when it is grown anaerobically, minimal ethanol is produced along with mixed acids, (fermentative growth on 25 mM glucose yielded 6.5 mM ethanol, 8.2 mM acetate, 6.5 mM lactate, 0.5 mM succinate, and about 1 mM formate leaving 10.4 mM residual glucose) Brau & Sahm (1986a) Arch. Microbiol. 144:296-301, (1986b) Arch. Microbiol. 146:105-110. When the genes encoding alcohol dehydrogenase II (adhB) and pyruvate decarboxylase (pdc) cloned from Z. mobilis are introduced and expressed into in E. coli, the initial concentration of 25 mM glucose was completely converted yielding up to 41.5 mM ethanol while almost forming no acids. This work has been expounded upon by other researchers. (Conway et al. (1987a) J. Bacteriol. 169:2591-2597; Neale et al. (1987) Nucleic Acids Res. 15:1752-1761; Ingram and Conway [1988] Appl. Environ. Microbiol. 54:397-404; Ingram et al. (1987) Appl. Environ. Microbiol. 53:2420-2425.) The extent of ethanol production under anaerobic and aerobic conditions was directly related to the level of expression of the Z. mobilis ethanologenic gene. Therefore, using appropriate transcription regulatory nucleotide sequences, a signal enzyme construct can be designed, that corresponds to the ethanologenic heterologous construct and then used poise the culture to improve the rate and quantity of ethanol production.

This technique is not limited to E. coli, since subsequent studies demonstrated the general applicability of this approach by the use of two other enteric bacteria, Erwinia chrysanthemi and Klebsiella planticola, to increase ethanol yields from hexoses, pentoses, and sugar mixtures. (Tolan and Finn. Appl. Environ. Microbiol. 53:2033-2038, 2039-2044, 1987; Beall et al., 1993; Ingram and Conway, 1988; Wood and Ingram, 1992.)

2.12 Synthetic Pathways

The term synthetic pathway includes natural, pre-existing pathways that generate secondary metabolites, also known as natural products, such as aliphatic, aromatic, and heteroaromatic organic acids, alkaloids, terpenoids, polyketides, phenols, iridoids, steroids, saponins, peptides, ethereal oils, resins and balsams. Additionally, a synthetic pathway also includes pathways introduced either whole or in part, into an organism through genetic engineering, cell fusion, conjugation, or other means. For example the introduction of an ethanologenic pathway in E. coli through the use of plasmids encoding the heterologous proteins from Z. mobilis for alcohol dehydrogenase II and pyruvate decarboxylase. Or the engineering of a tepenoid pathway in E. coli, through the expression of a synthetic amorpha-4,11-diene synthase gene derived from the plant Artemisia annua L. coupled with the mevalonate isoprenoid pathway from S. cerevisiae. (Martin, V. J. J. et al. Engineering a mevalonate pathway in Escherichia coli for production of terpenoids. Nature Biotech. 21:796-802, 2003; US Pat. Application Publication 2004/0005678 A1.)

### 3. Reporter Constructs

3.1. Method of Making Luciferase Expression Vectors for Use as Signal Enzymes

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Ausubel, F. M., et al., Current Protocols in Molecular Biology, John Wiley and Sons, Inc., Media, Pa. (1995.); Sambrook, J., et al., Molecular Cloning: A Laboratory Manual, Third Edition, Cold Spring Harbor Laboratory (Cold Spring Harbor, N.Y.) (2001).)

According to the present invention, a signal enzyme construct comprising of an expression cassette with the transcription regulatory nucleotide sequence for the gene of interest, operatively linked to the reporter gene and associated regulatory sequences and linkers, is inserted into an appropriate vector, that is then used to transform the intended host.

In one aspect this invention provides reporter constructs that are useful for monitoring the production of a target of a biochemical pathway in an organism. In certain embodiments, these constructs are used to provide such information in real time during culture of microorganisms. The constructs include recombinant nucleic acid molecules comprising transcription regulatory nucleotide sequences, e.g., promoters, operatively linked to a gene encoding a light-emitting reporter, wherein the transcription regulatory nucleotide sequences also regulate expression of an enzyme whose expression reports on the production of the target.

This invention contemplates, in particular, two embodiments of this system. In a first embodiment, the reporter construct is separate from the host gene and its transcription regulatory nucleotide sequences. The organism thus contains parallel regulatory constructs: One controlling expression of the enzyme and a copy controlling expression of the reporter. Because the transcription regulatory nucleotide sequences are the same, the expression level of the reporter mirrors the expression level of the signal enzyme in the system. The term “transcription regulatory nucleotide sequence” encompasses all nucleotide sequences that are responsible for the control of the expression of a gene. This includes promoter and enhancer sequences, and sequences where gene repressor proteins and gene activator proteins bind. It further includes regions where primary response proteins bind to activate the transcription of secondary response proteins. Furthermore, the term “transcription regulatory nucleotide sequence” encompasses modified nucleotide sequences that retain transcriptional regulatory activity. Additionally, the term “transcription regulatory nucleotide sequence” includes homologous transcription regulatory nucleotide sequences from other organisms, so that if the homologous sequence is substituted for the native sequence it will function in a similar manner.

In a second embodiment, the reporter is coupled to the native transcription regulatory nucleotide sequences so that the gene encoding the signal enzyme and the gene encoding the reporter are under control of the same nucleic acid segment.

3.2. Transcription Regulatory Nucleotide Sequences

The transcription regulatory nucleotide sequences for signal enzymes must be compatible with the intended host. According to the present invention, the most preferred transcription regulatory nucleotide sequences are those from the host organism. For the monitoring of the expression of acidogenic and solventogenic genes of C. acetobutylicum, the majority of the transcription regulatory nucleotide sequences for these genes are readily available. See Table 2. Through the analysis of the transcription regulatory nucleotide sequences, the appropriate primers can be designed so that the transcription regulatory nucleotide sequence of interest can be cloned from genomic DNA by use of the technique of polymerase chain reaction (PCR). The sequences of transcription regulatory for genes that are not listed in Table 2 can be identified through the use of computational methods utilizing the sequenced genome of C. acetobutylicum ATCC 824. (Paredes, C. J. et al. Transcriptional organization of the Clostridium acetobutylicum genome, Nuc. Acids Res. 32:1973-1981) Alternatively, since the sequences of the acidogenic and solventogenic genes are known and available through internet based services such as TIGR or the National Center for Biotechnology Information (NCBI, www.ncbi.nlm.nih.gov), the transcription regulatory nucleotide sequences can be identified through standard molecular biology techniques such as cDNA primer extension using primers derived from the gene sequences of interest coupled with reverse transcription.

TABLE 2
Sources for Transcription Regulatory Nucleotide Sequences
for Select Genes of C. acetobutylicum

| | | | IR | | |
|---|---|---|---|---|---|
| Gene ID | Direction | Annotation | Length | Description | Reference |
| CAC1742 | + | pta | 264 | Phosphotransacetylase | Boynton. Appl. |
| | | | | | Environ. |
| | | | | | Microbiol. 1996 |
| CAC1743 | + | askA | 11 | Acetate kinase | Boynton. Appl. |
| | | | | | Environ. |
| | | | | | Microbiol.1996 |
| CAC2708 | − | hbd | 104 | Beta-hydroxybutyryl- | Boynton. J. Bacteriol. |
| | | | | CoA dehydrogenase, | 1996 |
| | | | | NAD-dependent | |
| CAC2711 | − | bcd | 13 | Butyryl-CoA | Boynton. J. Bacteriol. |
| | | | | dehydrogenase | 199 |
| CAC2712 | − | crt | 175 | Crotonase (3- | Boynton. J. Bacteriol. |
| | | | | hydroxybutyryl-COA | 199 |
| | | | | dehydratase) | |
| CAC2873 | − | | 326 | Acetyl-CoA | Stim-Herndon. Gene. |
| | | | | acetyltransferase | 1995 |
| CAC3075 | − | buk | 27 | Butyrate kinase, BUK | Walter. Gene. 1993 |
| CAC3076 | − | ptb | 108 | Phosphate | Walter. Gene. 1993 |
| | | | | butyryltransferase | |
| CAC3298 | − | bdhB | 276 | NADH-dependent | Walter. J. Bacteriol. |
| | | | | butanol dehydrogenase B | 1992 |
| | | | | (BDH II) | |
| CAC3299 | − | bdhA | 147 | NADH-dependent | Walter. J. Bacteriol. |
| | | | | butanol dehydrogenase | 1992 |
| | | | | A (BDH I) | |
| CAP0035 | − | adhe | 476 | Aldehyde-alcohol | Fontaine. J. Bacteriol. |
| | | | | dehydrogenase ADHE1 | 2002 |
| CAP0078 | − | thil | 105 | Acetyl coenzyme A | Winzer. J. Mol. |
| | | | | acetyltransferase | Microbiol. Biotechnol., |
| | | | | (thiolase) | 2000 |
| CAP0162 | + | adhe1 | 666 | Aldehyde dehydrogenase | Nair. J. Bacteriol. 1994 |
| | | | | (NAD+) | Fischer. J. Bacteriol. |
| | | | | | 175: 6959-6969, 1993 |
| CAP0163 | + | ctfa | 63 | Butyrate-acetoacetate | Nair. J. Bacteriol. 1994 |
| | | | | COA-transferase | Fischer. J. Bacteriol. |
| | | | | subunit A | 175: 6959-6969, 1993 |
| CAP0164 | + | ctfb | 4 | Butyrate-acetoacetate | Nair. J. Bacteriol. 1994 |
| | | | | COA-transferase | Fischer. J. Bacteriol. |
| | | | | subunit B | 175: 6959-6969, 1993 |
| CAP0165 | − | adc | 232 | Acetoacetate | Gerischer. J. Bacteriol. |
| | | | | decarboxylase | 172, 1990 |
| | | | | | Gerischer. J. Bacteriol. |
| | | | | | 174: 426-433, 1992 |

a) Gene ID: Systematic gene code from TIGR.
b) Direction: Coding strand
c) Annotation: Gene symbol according to TIGR.
d) IR length: Length of the upstream Intergenic Region.
e) Description: Description of gene function.

3.3. Light Producing Molecules

The light producing molecules useful in the practice of the present invention may take any of a variety of forms, depending on the application. They share the characteristic that they are luminescent, that is, that they emit electromagnetic radiation in ultraviolet (UV), visible and/or infra-red (IR) from atoms or molecules as a result of the transition of an electronically excited state to a lower energy state, usually the ground state. Examples of light producing molecules include photoluminescent molecules, such as fluorescent molecules, chemiluminescent compounds, phosphorescent compounds, and bioluminescent molecules.

In certain embodiments, the light-emitting reporter is self-contained. As used herein, a light-emitting reporter is “self-contained” if it produces light without the addition of exogenous organic substrate. Thus, for example, fluorescent reporters are “self-contained.” The lux operon, which produces microbial luciferase, also produces a self-contained reporter in that it contains enzymes to produce the necessary substrate. By contrast, the luc gene, which produces a mammalian luciferase, requires the addition of a substrate such as luciferin and frequently ATP in order for there to be bioluminescence. Therefore, it is not self-contained. Self-contained reporters provide certain advantages in the methods of this invention because the addition of exogenous substrate can be expensive and introduce inefficiencies into monitoring and regulating the state of the culture.

3.3.1. Bioluminescent Proteins

Bioluminescent molecules are distinguished from fluorescent molecules in that they do not require the input of radiative energy to emit light. Rather, bioluminescent molecules utilize chemical energy, such as ATP, to produce light. An advantage of bioluminescent molecules, as opposed to fluorescent molecules, is that there is virtually no background in the signal. The only light detected is light that is produced by the exogenous bioluminescent molecule. In contrast, the light used to excite a fluorescent molecule often results in background fluorescence that interferes with signal measurement.

Several types of bioluminescent molecules are known. They include the luciferase family (de Wet, J. R, et al., Firefly luciferase gene: structure and expression in mammalian cells. Mol. Cell. Biol. 7:725-737, 1987) and the aequorin family (Prasher, et al. Cloning and expression of the cDNA coding for Aequorin, a bioluminescent calcium-binding protein. Biochem Biophys Res Commun 126: 1259-1268, 1985). Members of the luciferase family have been identified in a variety of prokaryotic and eukaryotic organisms. Prokaryotic luciferase is encoded by two subunits (luxAB) of a five gene complex that is termed the lux operon (luxCDABE). The remaining three genes comprise the luxCDE subunits and code for the fatty acid reductase responsible for the biosynthesis of the aldehyde substrate used by luciferase for the luminescent reaction.

Eukaryotic luciferase (“luc”) is typically encoded by a single gene (de Wet, J. R., et al., Proc. Natl. Acad. Sci. U.S.A. 82:7870-7873, 1985; de Wet, J. R, et al., Mol. Cell. Biol. 7:725-737, 1987). An exemplary eukaryotic organism containing a luciferase system is the North American firefly Photinus pyralis. Firefly luciferase has been extensively studied, and is widely used in ATP assays. cDNAs encoding luciferases (lucOR) from Pyrophorus plagiophthalamus, another species of click beetle, have been cloned and expressed. (Wood, et al. Complementary DNA coding click beetle luciferases can elicit bioluminescence of different colors. Science 244:700-702, 1989.) This beetle is unusual in that different members of the species emit bioluminescence of different colors. Four classes of clones, having 95-99% homology with each other, were isolated. They emit light at 546 nm (green), 560 nm (yellow-green), 578 nm (yellow) and 593 nm (orange).

Luciferases, as well as aequorin-like molecules, require a source of energy, such as ATP, NAD(P)H, a substrate to oxidize, such as luciferin (a long chain fatty aldehyde) or coelentrizine and oxygen. With the lux operon, the genes encoding the enzyme that synthesizes the aldehyde substrate are expressed contemporaneously with luciferase. In cells transformed with a lux signal enzyme construct, oxygen is the only extrinsic requirement for bioluminescence. Such constructs are self-contained, in the sense that no exogenous compounds need to be added other than oxygen. In contrast, cells transformed with a luc signal enzyme will require the addition of an exogenous substrate and frequently ATP in order for there to be luminescence.

The plasmid construct, encoding the lux operon obtained from the soil bacterium Photorhabdus luminescens, formerly Xenorhabdus luninescens (Frackman, et al., Cloning, organization, and expression of the bioluminescence genes of Xenorhabdus luninescens. J. Bacteriol. 172″5767-5773, 1990), confers on transformed E coli optimal bioluminescence at 37° C. (Xi, et al. Cloning and nucleotide sequences of lux genes and characterization of Luciferase of Xenorhabdus luninescens from a human wound. J. Bacter. 173:1399-1405, 1991.) The sequence is available from GenBank under the accession number M90092. In contrast to luciferase from P. luminescens, other luciferases isolated from luminescent prokaryotic and eukaryotic organisms have optimal bioluminescence at lower temperatures. (Campbell, A. K. Chemiluminescence, Principles and Applications in Biology and Medicine. Ellis Horwood, Chichester, UK, 1988,)

To facilitate the expression of P. luminescens lux in C. acetobutylicum, the nucleotide sequence of the wild type lux operon (luxCDABE) was reengineered to have a AT content of 69%. This was accomplished by taking advantage of the degeneracy of the genetic code so that codons that include C or G at degenerate positions could be replaced with codons that encode the same amino acid, but have a A or T in the degenerate positions. The sequences of the individual genes of the C. acetobutylicum optimized lux operon, along with their corresponding amino acid sequences are given in SEQ ID NO: 1-10. One can similarly modify other light emitting proteins so that they are optimized for expression in C. acetobutylicum and other organisms with high AT content in the range of 60-80%.

A variety of other luciferase encoding genes have been identified including, but not limited to, the following: Sherf, B. A., and Wood, K. V., U.S. Pat. No. 5,670,356; Kazami, J., et al., U.S. Pat. No. 5,604,123; Zenno, S., et al, U.S. Pat. No. 5,618,722; Wood, K. V., U.S. Pat. No. 5,650,289; Wood, K. V, U.S. Pat. No. 5,641,641; Kajiyama N., and Nakano, E., U.S. Pat. No. 5,229,285; Cormier, M. J., and Lorenz, W. W., U.S. Pat. No. 5,292,658; Cormier, M. J., and Lorenz, W. W., U.S. Pat. No. 5,418,155; de Wet, J. R., et al, Molec. Cell. Biol. 7:725-737, 1987; Tatsumi, H. N., et al, Biochim. Biophys. Acta 1131:161-165, 1992; and Wood, K. V., et al, Science 244:700-702, 1989, all herein incorporated by reference. Such luciferase encoding genes may be modified by the methods described herein to produce polypeptide sequences and/or expression cassettes useful, for example, in Gram-positive microorganisms.

3.3.2. Fluorescent Proteins

Fluorescence is the luminescence of a substance from a single electronically excited state, which is of very short duration after removal of the source of radiation. The wavelength of the emitted fluorescence light is longer than that of the exciting illumination (Stokes' Law), because part of the exciting light is converted into heat by the fluorescent molecule.

Background fluorescence and stray light from the excitatory illumination source may complicate the use of fluorescent molecules. Shielding of the illumination source may be required along with the use of an excitation filter, to block the majority of photons having a wavelength similar to that of the photons emitted by the fluorescent moiety. Similarly a barrier filter can be used with the detector to screen out most of the photons having wavelengths other those emitted by the fluorescent molecules. Alternatively, a laser producing high intensity light near the appropriate excitation wavelength, but not near the fluorescence emission wavelength, can be used to excite the fluorescent moieties.

Fluorescent molecules include small molecules, such as fluorescein, as well as fluorescent proteins, such as green fluorescent protein (GFP) (Chalfie, et al., Morin, et al.), lumazine, and yellow fluorescent proteins (YFP), (O'Kane, et al., Daubner, et al.) In nature, fluorescent proteins are often found associated with luciferase and function as the ultimate bioluminescence emitter in these organisms by accepting energy from enzyme-bound, excited-state oxyluciferin (Ward et al. (1979) J. Biol. Chem. 254:781-788; Ward et al. (1978) Photochem. Photobiol. 27:389-396; Ward et al. (1982) Biochemistry 21:4535-4540.) They can be used in the present system to increase the detector sensitivity to the bioluminescence generating system and to also shift the wavelength of the emitted light to a more appropriate wavelength for detection purposes.

The best characterized GFPs are those isolated from the jellyfish species Aequorea, particularly Aequorea victoria (A. victoria) and Aequorea forskalea and the sea pansy Renilla reniformis. (Ward et al. Biochemistry 21:4535-4540; 1982; Prendergast et al. Biochemistry 17:3448-3453, 1978.) In A. victoria, GFP absorbs light generated by aequorin upon the addition of calcium and emits a green fluorescence with an emission wavelength of about 510 nm. (Ward et al. Photochem. Photobiol. Rev 4:1-57, 1979.)

Aequorea GFP encodes a chromophore intrinsically within its protein sequence, obviating the need for external substrates or cofactors and enabling the genetic encoding of strong fluorescence. (Ormo, M., et al. Crystal structure of the Aequorea victoria green fluorescent protein. Science 273:1392-1395, 1996.) The chromophore is centrally located within the barrel structure and is completely shielded from exposure to bulk solvent. Mutagenesis studies have generated GFP variants with new colors, improved fluorescence and other biochemical properties.

DNA encoding an isotype of A. victoria GFP has been isolated and its nucleotide sequence has been determined. (Prasher (1992) Gene 111:229-233.) Recombinantly expressed A. victoria GFPs retain their ability to fluoresce in vivo in a wide variety organisms, including bacteria (e.g., see Chalfie et al. (1994) Science 263:802-805; Miller et al. (1997) Gene 191:149-153), yeast and fungi (Fey et al. (1995) Gene 165:127-130; Straight et al. (1996) Curr. Biol. 6:1599-1608; Cormack et al. (1997) Microbiology 143:303-311).

Patents relating to A. victoria GFP and mutants thereof include the following: Chalfie, M., and Prasher, D. U.S. Pat. No. 5,491,084; Tsien, R., and Heim, R. U.S. Pat. No. 5,625,048; Tsien, R., and Heim, R. U.S. Pat. No. 5,777,079; Zolotukhin, S., et al. U.S. Pat. No. 5,874,304; Anderson, M., and Herzenberg, L. A. U.S. Pat. No. 5,968,738; Cormack, B. P., et al. U.S. Pat. No. 5,804,387; Tsien, R., and Heim, R. U.S. Pat. No. 6,066,476; Chalfie, M., and Prasher, D. U.S. Pat. No. 6,146,826; and Tsien, R., et al. U.S. Pat. No. 7,005,511. Patents relating to such fluorescent encoding genes may be modified by the methods described herein to produce polypeptide sequences and/or expression cassettes useful, for example, in Gram-positive microorganisms.

3.4. Colorimetric or Fluorometric Reactions

As an alternative to light producing molecules, enzymes that catalyze colormetric or fluorometric reactions or synthesis colormetric or fluorometric substrates are also useful in the practice of the present invention and may take any of a variety of forms, depending on the application. The use of reporter constructs that encode for enzymes that catalyze colormetric or fluorometric reactions may be advantageous when used to analyze complex samples such as fermentation broth, because enzymes have exquisite specificity for their substrates. Additionally, the signal strength of the colormetric or fluorometric reactions increases over time as more substrate is converted to the colormetric or fluorometric product.

One colormetric enzyme contemplated for use as a signal enzyme is β-galactosidase produced by the bacterial gene lacZ. This enzyme cleaves the colorless substrate X-gal (5-bromo-4-chloro-3-indolyl-b-D-galactopyranoside) into galactose and a blue insoluble product. A bacterial lacZ gene can be used in C. acetobutylicum since it was shown that C. acetobutylicum does not possess a β-galactosidase. (Yu, P.-L., et al. Differential induction of β-galactosidase and phospho-β-galactosidase activities in the fermentation of whey permeate by Clostridium acetobutylicum. Appl. Microbiol. Biotechnol. 26:254-257. 1987.) The lacZ gene from Thermoanaerobacterium thermosulfurigenes, a low G+C content organism with similar codon usage as of Clostridial species was demonstrated to function well as a reporter in C. acetobutylicum. (Burchhardt, G., and H. Bahl. Cloning and analysis of the β-galactosidase-encoding gene from Clostridium thermosulfurogenes EM1. Gene 106:13-19, 1991.) Other enzymes that can be used include, the gusA gene encoding β-glucuronidase (Girbal, L., et al. Development of a sensitive gene expression reporter system and an inducible promoter-repressor system for Clostridium acetobutylicum. Appl. Environ. Microbiol. 69:4985-4988, 2003), and possibly the eglA gene encoding a β-1,4-endoglucanase from Clostridium saccharobutylicum (Quixley, K. et al. Construction of a reporter gene vector for Clostridium beijerinckii using a Clostridium endoglucanase gene. J. Mol. Microbiol. Biotechnol. 2:53-57, 2000).

3.5. Expression Cassettes

The desired transcription regulatory nucleotide sequence for an enzyme to be monitored is operably linked to a gene encoding a reporter enzyme along with the appropriate translational regulatory elements (e.g., Gram-positive Shine-Dalgarno sequences), short, random nucleotide sequences, and selectable markers, to form what is termed an expression cassette. The methodologies utilized in making the individual components of an expression cassette and in assembling the components are well known in the art of molecular biology (see, for example, Ausubel, F. M., et al., or Sambrook, et al.) in view of the teachings of the specification. Examples of expression cassettes useful in the present invention include the gusA reporter cassette (Girbal, L., et al. supra) and the lacZ reporter cassette (Tummala, S. B. et al. Development and characterization of a gene expression reporter system for Clostridium acetobutylicum ATCC 824, Appl. Envir. Mircobiol. 65:3793-3799, 1999). A preferred embodiment of this invention uses an expression cassette with P. luminescens lux in the wild type arrangement of CDABE that has been optimized for expression in C. acetobutylicum and has Gram-positive bacterial Shine-Dalgarno sequences 5′ to each lux gene. SEQ ID NO: 11. Another preferred embodiment uses an expression cassette with the P. luminescens lux genes that have been optimized for expression in C. acetobutylicum and have Gram-positive bacterial Shine-Dalgarno sequences 5′ to each lux gene but are arranged in a non-wild type sequence such as luxABCDE (U.S. Pat. No. 6,737,245).

The bacterial lux operon is self-contained as the operon contains the genes for the endogenous production of an aldehyde substrate, unlike the eukaryotic luc operon. Therefore, the contemporaneous coproduction of luciferase and endogenous aldehyde substrate allows for real time measurement of bioluminescence without the need to add exogenous aldehyde before monitoring the bioluminescent signal strength. A luxAB construct could, however, be utilized and an aldehyde substrate added prior to measurement of bioluminescence as is required with signal enzyme constructs utilizing the luc operon. One preferred embodiment of the present invention uses a luciferase expression cassette wherein the lux operon from P. luminescens is operationally linked to the appropriate transcription regulatory nucleotide sequence for an enzyme in a fermentative pathway of C. acetobutylicum in a manner analogous to U.S. Pat. No. 6,737,245. Another preferred embodiment of this invention uses an expression cassette with a gene encoding a fluorescent protein operationally linked to the appropriate transcription regulatory nucleotide sequence for an enzyme in a fermentative pathway of C. acetobutylicum

3.6. Shuttle Vectors

Expression cassettes are then inserted into “shuttle vectors”, plasmids that can replicate in two or more hosts. A shuttle vector to be used with gram negative and gram positive organisms requires the shuttle vector to contain an origin of replication from each class. Examples of shuttle vectors include the pAUL-A vector (Chakraborty, et al. (1992) J. Bacteriol. 174:568 574), pMK4 and pSUM series (U.S. Pat. No. 6,737,245), and pIMP1 (Mermelstein, L. D., et al. Bio/Technology 10:190-195, 1992). Other vectors are well known to those skilled in the art and are readily available from catalogs.

3.7. Chromosomal Integration

Instead of transforming an organism with a plasmid, a signal enzyme can be integrated into a chromosome of the host. Use of chromosomal integration of the reporter construct offers several advantages over plasmid-based constructions, including greater stability, and the elimination of the use of antibiotics to maintain selective pressure on the organisms to retain the plasmids. One method to achieve chromosomal integration uses a DNA fragment that contains the desired gene upstream from an antibiotic resistance gene such as the chloramphenicol gene and a fragment of homologous DNA from the target organism. This DNA fragment can be ligated to form circles without replicons and used for transformation. For example, the pfl gene can be targeted in the case of E. coli, and short, random Sau3A fragments can be ligated in Klebsiella to promote homologous recombination. In this way, ethanologenic genes have been integrated chromosomally in E. coli. (Ohta et al. Appl. Environ. Microbiol. 57: 893-900, 1991.)

The copy number of the integrated reporter can be controlled by the concentration of the antibiotic used in the selection process. For example, when a low concentration of antibiotics is used for selection, clones with single copy integrations are found, albeit at very low frequency. While this may be disadvantageous for many genes, a low copy number for luciferase may be ideal given the high sensitivity of the detectors employed in light measurement. Higher level expression can be achieved in a single step by selection on plates containing much higher concentrations of antibiotic.

Another method for chromosomal integration uses a transposable element such as a transposon, that provides for the introduction of an engineered cassette.

3.8. Signal Enzymes that Parallel the Regulatory Control of the Monitored Enzymes.

The expression of signal enzymes on shuttle vectors within a transformed host will naturally parallel that of the native enzyme that is to be monitored, since there will be two independent transcription regulatory nucleotide sequences present. Chromosomal integration will also result in parallel regulatory control, unless one is able to introduce the signal enzyme sequence in-line with the native gene.

3.9. Signal Enzymes Having Regulatory Control in-Line with the Monitored Enzymes

One way to place a signal enzyme under the same regulatory control as that of the native enzyme is to select the use of an operon located on an endogenous plasmid, like sol located on the pSOL1 megaplasmid. Here, the plasmid can be isolated, the operon excised and replaced by an expression cassette containing a new operon wherein the reporter gene is inserted in-line with the native gene to be monitored. Following transformation and amplification in an appropriate host, the plasmid can then be isolated and then used to transform a pSOL1plasmid deficient strain of C. acetobutylicum.

3.10. Transformation of C. acetobutylicum

Numerous methods for the introduction of signal enzyme constructs into cells or protoplasts of cells are known to those of skill in the art and include, but are not limited to, the following: lipid-mediated transfer (e.g., using liposomes, including neutral and cationic lipids), direct injection (e.g., microinjection), cell fusion, microprojectile bombardment (e.g., biolistic methods, such as DNA particle bombardment), co-precipitation (e.g., with calcium phosphate, or lithium acetate), DEAE-dextran- or polyethylene glycol-mediated transfer, viral vector-mediated transfer, electroporation and conjugation.

Electroporation is the preferred method of transforming C. acetobutylicum. Ideally, electrocompetent C. acetobutylicum cells prepared from mid-logarithmic growth phase are used. Following electroporation, cells are incubated at 37° C. in an appropriate broth, like 2×YT broth while under a nitrogen atmosphere. Following a recovery period, the cells are transferred to an anaerobic glovebox, and serial dilutions are then plated on nutrient plates like 2×YT agar plates that are supplemented with the requisite antibiotic concentration.

3.11. Detection of Clones with Luciferase Containing Signal Enzyme Constructs

Colonies of microorganisms that contain signal enzyme constructs derived from the complete luxCDABE operon, can be identified by manual visual inspection in a darkened room or by the use of an image detection system such as one that incorporates a charge coupled device (CCD) camera. Since oxygen is required for the bioluminescence reaction, plates may need to be exposed to low concentrations of oxygen in order to detect positive colonies. The expression cassettes derived from luc and luxAB require the addition of an exogenous substrate in order to produce light. In a preferred embodiment of the present invention, the substrate is aldehyde. When administered to cells, aldehyde may be applied in the atmosphere surrounding the culture media as a vapor or directly to the culture media.

### 4. Cells and Cultures

The use of signal enzymes is applicable for the monitoring of all types of fermentative or synthetic pathways. The hosts may by “wild type” wherein they natively produce the desired target, or they may have already undergone mutagenesis and positive selection to overproduce the desired target. Alternatively, the host can be previously engineered to express enzymes required for the desired fermentative or synthetic pathway. This can be in the form of overexpressing the native enzymes required for the fermentative or synthetic pathways or the expression of heterologous enzymes required for a fermentative or synthetic pathway. Additionally, signal enzymes can be introduced simultaneously into the host cells with either native or heterologous fermentative or synthetic pathway enzymes. With simultaneous introduction, the signal enzymes can be on the same operon as the introduced fermentative or synthetic pathway enzymes or the signal enzymes can be located on different operons. Furthermore, the host can also be genetically modified so that expression of a necessary enzyme for a competing fermentative or synthetic pathway is down regulated or negated, thereby forcing substrate down the fermentative or synthetic pathway of interest. With C. acetobutylicum, wild types strains contemplated for use with this invention include ATCC 824 and ATCC 43084 from the American Tissue Culture Collection (ATCC) and DSM 792 and DSM 1731 from the Deutsche Sammlung von Mikroorganismen and Zellkulturen GmbH, Germany. High butanol producing mutants of C. acetobutylicum contemplated for use with this invention include strains such as ATCC 39058, and ATCC 55025 (U.S. Pat. No. 5,192,673) from ATCC. Another high producing strain contemplated for use with this invent is B643. (Contag, P. R., et al, Cloning of a lactate dehydrogenase gene from Clostridium acetobutylicum B643 and expression in Escherichia coli. Appl. Environ. Microbiol. 56:3760-3765, 1990.) A further high producing mutant contemplated for use with this invention is B18 that was derived from B643, above. Enzymes anticipated to be overexpressed in C. acetobutylicum for the production of butanol include butyraldehyde dehydrogenase and butanol dehydrogenase. Enzymes of competing fermentative pathways anticipated to by down regulated or deleted in C. acetobutylicum include pyruvate decarboxylase, lactate dehydrogenase and acetate kinase.

Strains of C. beijernickii contemplated for use with this invention include, strains ATCC 25752, ATCC 51743 and BA101, ATCC PTA 1550 (U.S. Pat. No. 6,358,717 and U.S. application Ser. No. 10/945,551). Other species of Clostridia contemplated for use with this invention include C. saccharobutylicum strain ATCC BAA-117; and C. puniceum strain ATCC 43978.

The cell cultures of this invention are characterized in that they produce a target of a synthetic or fermentative pathway in commercially valuable quantities and they also produce a light emitting reporter that signals the status of target production.

In certain embodiments, commercially valuable quantities of a target include those targets produced in 100 l fermentors. In other embodiments, commercially valuable quantities of a target are produced in fermentors with 100 to 500 l capacity. In still further embodiments, commercially valuable quantities of a target are produced in fermentors of 500 l to 1,000 l capacity. In still other embodiments, commercially valuable quantities of a target are produced in fermentors of 1,000 l to 2000 l capacity. In certain other embodiments, commercially valuable quantities of a target are produced in fermentors with 2,000 l to 5,000 l capacity. In other embodiments, commercially valuable quantities of a target are produced in fermentors with 5000 l to 10,000 l capacity. In still other embodiments, commercially valuable quantities of targets are produced in fermentors with 10,000 l to 50,000 l capacity. In certain other embodiments, commercially valuable quantities of targets are produced in fermentors with 50,000 l to 200,000 l capacity. In still further embodiments, commercially valuable quantities of targets are produced in fermentors with 200,000 l to 400,000 l capacity. In certain embodiments, commercially valuable quantities of targets are produced in fermentors with 400,000 l to 800,000 l capacity. In still other embodiments, commercially valuable quantities of targets are produced in fermentors with 800,000 l to 2,000,000 l capacity. In certain embodiments, commercially valuable quantities of targets are produced in fermentors with 2,000,000 l to 4,000,000 l capacity. In other embodiments, commercially valuable quantities of targets are produced in fermentors with 4,000,000 l to 8,000,000 l capacity.

4.1 Substrates

The substrates of the present invention are carbon-based compounds that can be converted enzymatically to intermediate compounds. As used herein, the term “carbon substrate: refers to material containing at least one carbon atom which can be enzymatically converted into an intermediate for subsequent conversion into the desired carbon target. Exemplary carbon substrates include, but are not limited to biomass, starches, dextrins and sugars.

As used herein, “biomass” refers to cellulose- and/or starch-containing raw materials, including but not limited to wood chips, corn stover, rice, grasses, forages, perrie-grass, potatoes, tubers, roots, whole ground corn, grape pomace, cobs, grains, wheat, barley, rye, milo, brans, cereals, sugar-containing raw materials (e.g., molasses, fruit materials, sugar cane, or sugar beets), wood, and plant residues. Indeed, it is not intended that the present invention be limited to any particular material used as biomass. In preferred embodiments of the present invention, the raw materials are starch-containing raw materials (e.g., cobs, whole ground corns, corns, grains, milo, and/or cereals, and mixtures thereof). In particularly preferred embodiments, the term refers to any starch-containing material originally obtained from any plant source including food processing waste such as almond and other nut shells, prunings and clippings from orchards and vineyards, and cropped fruit like grapes.

As used herein, “starch” refers to any starch-containing materials. In particular, the term refers to various plant-based materials, including but not limited to wheat, barley, potato, sweet potato, tapioca, corn, maize, cassava, milo, rye, and brans. Indeed, it is not intended that the present invention be limited to any particular type and/or source of starch. In general, the term refers to any material comprised of the complex polysaccharide carbohydrates of plants, comprised of amylose, and amylopectin, with the formula (C₆H₁₀O₅)ₓ, wherein “x” can be any number.

As used herein, “cellulose” refers to any cellulose-containing materials. In particular, the term refers to the polymer of glucose (or “cellobiose”), with the formula (C₆H₁₀O₅)ₓ, wherein “x” can be any number. Cellulose is the chief constituent of plant cell walls and is among the most abundant organic substances in nature. While there is a β-glucoside linkage in cellulose, there is an α-glucoside linkage in starch. In combination with lignin, cellulose forms “lignocellulose.”

As used herein, “hemicellulose” refers to any hemicellulose-containing materials. In particular, the term refers to hetropolymers with xylosyl-, glucosyl-, galactosyl-, arabinosyl- or mannosyl-residues.

Suitable substrates include, but are not limited to processed materials that contain constituents which can be converted into sugars (e.g. cellulosic biomass, glycogen, starch, and various forms thereof, such as corn starch, wheat starch, corn solids, and wheat solids). During the development of the present invention good results were obtained with corn, sorghum, and wheat starch, although other sources, including starches from other grains and tubers (e.g., sweet potato, potato, rice and cassava starch) also find use with the present invention. Various starches are commercially available.

Fermentable sugars can be obtained from a wide variety of sources, including lignocellulosic material. Lignocellulose material can be obtained from lignocellulosic waste products (e.g., plant residues and waste paper). Examples of suitable plant residues include but are not limited to any plant material such as stems, leaves, hull, husks, cobs and the like, as well as corn stover, begasses, wood, wood chips, wood pulp and sawdust. Examples of waste paper include but are not limited to discarded paper of any type (e.g., photocopy paper, computer printer paper, notebook paper, notepad paper, typewritter paper, and the like), as well as newspapers, magazines, cardboard, and paper-based packaging material.

An alternate fermentative substrate is dairy whey, a solution that after casein removal contains roughly 4 to 5% lactose, a disaccharide that can be directly fermented by Clostridia. This substrate is widely available and the fermentative use of whey for solvent production would solve the current whey disposal problem.

Recently, extruded organic waste collected from residential garbage was shown to contain 28 to 39% total sugar that is fermentable by C. acetobutylicum for solvent production. (Lopez-Contreras, A. M., Utilisation of saccharides in extruded domestic organic waste by Clostridium acetobutylicum ATCC824 for production of acetone, butanol, and ethanol. Appl. Microbiol. Biotechnol. 54:162-167, 2000.) The diversion of residential organic waste away from disposal sites will help alleviate pressure on existing disposal sites and provides a higher value alternative to composting.

The conditions for converting sugars to ethanol are known in the art. Generally, the temperature is between about 25° C. and 35° C. (e.g., between 25° C. and 35° C. and more particularly at 30° C.). Useful pH ranges for the conversion medium are provided between 4.0 and 6.0, between 4.5 and 6.0, and between pH 5.5 and 5.8. However, it is not intended that the present invention be limited to any particular temperature and/or pH conditions as these conditions are dependent upon the substrate(s), enzyme(s), intermediate(s), and/or target(s) involved.

4.2 Media and Carbon Substrates

The conversion media in the present invention must contain suitable carbon substrates. Suitable carbon substrates include, but are not limited to biomass, monosaccharides (e.g., glucose and fructose), disaccharides (e.g., lactose and sucrose), oligosaccharides (e.g., starch and cellulose), as well as mixtures thereof, and unpurified mixtures from renewable feedstocks such as cheese whey permeate, cornsteep liquor, sugar beet molasses, and barley malt. In additional embodiments, the carbon substrate comprises one-carbon substrates such as carbon monoxide, or methanol for which metabolic conversion into key biochemical intermediates has been demonstrated.

Glycerol production from single carbon sources (e.g., methanol, formaldehyde, or formate) has been reported in methylotrophic yeasts (Yamada et al. Agric. Biol. Chem., 53:541-542, 1989) and in bacteria (Hunter et al. Biochem., 24:4148-4155, 1985). These organisms can assimilate single carbon compounds, ranging in oxidation state from methane to formate, and produce glycerol. In some embodiments, the pathway of carbon assimilation is through ribulose monophosphate, through serine, or through xylulose-monophosphate. (Gottschalk, Bacterial Metabolism, 2^{nd} Ed., Springer-Verlag, New York, 1986.) The ribulose monophosphate pathway involves the condensation of formate with ribulose-5-phosphate to form a 6-carbon sugar that becomes fructose and eventually the 3-carbon product glyceraldehyde-3-phosphate. Likewise, the serine pathway assimilates the one-carbon compound into the glycolytic pathway via methylenetetrahydrofolate.

In addition to the utilization of one and two carbon substrates, methyltrophic organisms are known to utilize a number of other carbon-containing compounds such as methylamine, glucosamine, and a variety of amino acids for metabolic activity. For example, methylotrophic yeast are known to utilize the carbon from methylamine to form trehalose or glycerol. (Bellion et al. in Murrell et al. (eds) 7^{th} Microb. Growth C1-Compd. Int. Symp., 415-432, Intercept, Andover, UK, 1993.) Similarly, various species of Candida metabolize alanine or oleic acid. (Sulter et al. Arch. Microbiol., 153:485-489, 1990.) Hence, the source of carbon utilized in the present invention encompasses a wide variety of carbon-containing substrates and is only limited by the requirements of the host organism.

Although it is contemplated that all of the above mentioned carbon substrates and mixtures thereof will find use in the methods of the present invention, preferred carbon substrates include monosaccharides, disaccharides, oligosaccharides, polysaccharides, and one-carbon substrates. In more particularly preferred embodiments, the carbon substrates are selected from the groups consisting of glucose, fructose, sucrose, and single carbon substrates such as methanol, and carbon monoxide. In a most particularly preferred embodiment, the substrate is glucose.

As known in the art, in addition to an appropriate carbon source, fermentation media must contain suitable nitrogen source(s), mineral salts, cofactors, buffers, and other components suitable for the growth of the cultures and promotion of the enzymatic pathway necessary for the production of the desire target (e.g., glycerol). In some embodiments, salts and/or vitamin B₁₂ or precursors thereof find use in the present invention.

### 5. Methods of Monitoring and Regulating

During growth and culture of microorganisms, the kinetics of various biochemical pathways change, shifting the rate of production of various targets. For example, in the batch culture of C. acetobutylicum, the initial production of acids, such as acetate and butyrate, decreases the pH of the culture, however, once the concentration of undissociated butyrate reaches 9 mM, a shift occurs wherein C. acetobutylicum reassimilates the secreted acids and switches to the production of solvents such as butanol and acetone. Butanol has a toxic effect upon the cells and its accumulation eventually inhibits the expression of the enzymes that produce it. By placing reporters at strategic points in various biochemical pathways one can monitor the status of these pathways and, if desired, one can “poise” the culture conditions to induce and maintain a state that produces the maximum amount of a product. In the case of an observed inhibitory effect of butanol on the culture, the removal of butanol from the fermentation broth can commence or water or culture media can be added to the fermentor to dilute the accumulated butanol below the inhibitory threshold.

The status of a biochemical pathway is signaled by the intensity of the signal being produced by the reporter. This, in turn, reflects the transcriptional activity of signal enzyme construct. Light emitting reporters are particularly attractive because they produce a signal in real time that correlates with the degree of gene expression providing immediate information regarding the status of a fermentative or synthetic pathway. The use of signal enzymes in C. acetobutylicum cultures allows culture conditions to be adjusted immediately to maintain or induce high productivity.

5.1. Detection of Light in a Culture

This invention contemplates several ways in which to measure light in a microbial culture. Conventionally fermentors can have one or more port holes positioned on the side of the tank so that the port hole is beneath the initial level of the fermentation broth. A means of detecting light such as a photomultiplier tube (PMT), or a CCD camera can then be mounted outside of a port hole outfitted with a clear window, but positioned to detect light that is emitted through the port hole window. Alternatively, an externally mounted PMT or CCD camera can be connected to a fiber optic cable or other type of light guide that is placed inside of the fermentor through a port hole or other opening prior to sterilization of the fermentor. The fiber optic cable may be attached to a flow cell engineered into the impeller of the fermentation agitator.

Light measurement can also be accomplished by placing the detector inside the fermentor. The detector may be mounted in a fixed position or tethered and rely on wiring to rely the signals to the data acquisition and analysis electronics. Alternately, the detector may use a wireless system of communication that in addition to the options of having the detector mounted in a fixed position or tethered, would allow for the detector to be free floating or to operate under its own power to move through the fermentation broth. The detector may further be designed as an integrated microfluidic chip with a CCD imager and a cooling element.

Additionally, a stream of the culture media can be continuously drawn off the fermentor and directed to a light detection apparatus. There the sample stream can be either intermittently or continuously passed through a flow cell positioned inside the light detection apparatus. Here, a mixing chamber can be place so that ATP or oxygen can be added to the sample stream if it is needed to enhance the luminescence of the media. Alternately, a diluent can be added to the sample in the mixing chamber to decrease the signal intensity if needed.

Furthermore, samples can be drawn off the fermentor periodically, through a sampling port either manually or automatically, and then analyzed for luminescence.

5.2. Processing of the Light Signal

An important aspect of the present invention is the use of a highly sensitive means to enable the rapid measurement of bioluminescence from fermentation broth so that the obtained signal can be used for real time monitoring and control of the culture. The device needs to be able to detect and count individual photons and accumulate the total count over time like in the manner of a scintillation counter. The most sensitive counting device employs a photomultiplying tube (PMT) wherein light entering the PMT excites electrons in the photocathode resulting in the emission of photoelectrons that as they are accelerated towards the detector unleash a growing cascade of electrons that are detected. Numerous PMTs are available from suppliers such as Hamamatsu. Spectrographic information can be obtained by employing light filters, gratings and other spectrographic devices in conjunction with the PMTs.

Less sensitive devices include charge coupled device (CCD) cameras. These can be cooled to reduce background noise or they can contain microchannel intensifiers that function in a manner analogous to a PMT to boast the signal generated by incident photons. An exemplary microchannel intensifier-based single-photon detection device is the C2400 series, available from Hamamatsu. Other potential counting technologies include integrating CCD, electron multiplying CCD, avalanche photodiodes and complementary metal oxide semiconductor (CMOS) image sensors.

Both PMTs and CCDs are available in modules for convenience that contain all the necessary power sources and electronic circuitry. For example a PMT module usually contains a high voltage power supply, voltage divider circuitry, signal conversion circuitry, photon counting circuitry, CPU interface and a cooling device integrated into a single package. Software is readily available that allows integration of the photon count signal with a computer thereby allowing the signal to be used in an algorithm for the monitoring and control a fermentation process.

5.3. Determining Status of the Biochemical Pathway: Computer Software

Determining the status of a biochemical pathway depends on the nature of signal enzyme on which the reporter reports. The signal can be positively or negatively correlated with the production of the target depending on whether the signal enzyme catalyzes a transformation toward the target or toward a branch leading either to another end product or to an intermediate that is recycled back to the pathway. Between these two alternatives, the absolute level of the signal provides information about the production of the desired product, and the kinetics of the signal, that is the change in intensity over time, also provides information about whether product production is increasing or decreasing. Additionally, the rate of change in the kinetics can also be calculated and used to monitor and control the fermentation.

While this information can be processed and acted upon by a person, in certain embodiments the information is processed by a computer. Thus, software of this invention will include code that receives as input data concerning the level of signal from each of the reporters, code that executes an algorithm that determines the state of the culture as a function of (at least) this level or level, and code that determines how the culture conditions should be changed to poise that culture at a desired state, and code that instructs the system to make the appropriate changes to the culture to achieve this condition, be it adjusting temperature, adding nutrients, removing a product from culture, decreasing the density of the culture, or any other change that will shift the culture to a desired state.

5.4. Regulating Pathway Activity in Culture

The ability to monitor enzyme expression and hence, activity along fermentative pathways, in real-time by the use of signal enzymes provides the operator or fermentation process controller with the ability to adjust conditions to “poise” the culture in a particular phase for maximum productivity of the desired end-product. One way to utilize the real time signaling capability of signal enzymes to control a culture is to adopt the real time signal methodologies used to control common high cell density E. coli fermentations. Here, cells are typically grown in batch mode to an intermediate cell density following which feeding strategies are initiated. The feeding strategies can be classified into two major categories: open-loop (non-feedback) and closed-loop (feedback). (U.S. Pat. No. 6,955,892.) The open-loop feeding strategies are typically pre-determined feed profiles for carbon/nutrient addition. Commonly used feed schedules include constant or increasing feed rates (constant, stepwise or exponential) in order to keep up with the increasing cell densities. While these simple pre-determined feed profiles have been applied successfully in certain cases, the major drawback is the lack of feed rate adjustment based on metabolic feedback from the culture. Therefore, the open-loop feeding strategies can fail by overfeeding or underfeeding the culture when it deviates from its “expected” growth pattern.

The closed-loop feeding strategies, on the other hand, typically rely on measurements that indicate the metabolic state of the culture. The two most commonly measured online variables for E. coli are dissolved oxygen (DO) concentration and pH. With DO monitoring, a rising DO signifies a reduction of oxygen consumption that in turn is based on nutrient limitation or depletion. When the DO rises above a threshold value or the rate of change is above a threshold value, the process controller will increase the nutrient feed rate. Conversely, when the DO drops below the desired set point or the rate of change is above a threshold value, the process control will reduce the nutrient feed rate to reflect metabolic demand. Similarly, changes in culture pH or the rate of change of a culture pH can be used alone or in combination with DO measurements to adjust the rate at which nutrient feed is added to the fermentor.

The redox potential is an alternate variable that can be measured and used to monitor and control a bacterial fermentation. The redox potential of the fermentation broth can be raised as needed through the addition of reducing agents. With a redox probe, trace oxygen concentrations in anaerobic cultures can be detected that are below the sensitivity of DO probes of <1 ppm.

Other methods of monitoring the metabolic activity of cultures include the analysis of fermentation broth and exhaust or off-gases. While both methods can be performed on-line, they cannot be performed in situ and will not provide information on the genetic expression of enzymes involved in the fermentative pathways. Rather, such analysis will only provide information on the general metabolic state of the culture.

Since signal enzymes provide real time status of the metabolic activity of the culture, the same process control algorithms used with DO and pH control of conventional high density cell culture systems can be adopted for use with signal enzymes systems. This would be particularly advantageous in the monitoring of anaerobic cultures where DO monitoring is impossible. Taking butanol production in C. acetobutylicum as an example, once the culture is firmly into the solventogenic phase, the majority of intermediates for butanol production will come from the continued metabolism of feedstock like glucose. Use of a signal enzyme towards the end of the butylic pathway such as bdhB, an aldehyde-alcohol dehydrogenase that reduces butyraldehyde to butanol, provides status as to the production of butanol and hence, the metabolic rate of the culture. The signal strength and rate of change of the signal strength can then be used to control the feed rate of the culture in much the same way as it is done by DO monitoring in E. coli cultures. This can be done in C. acetobutylicum batch culture by monitoring the initial expression of the signal enzyme as the culture starts to produce solvents. There will be an initial increase in the signal strength as organic acids from the acidogenic phase are reassimilated. As the concentration of these acids decrease, enzymatic activity will decrease in parallel signaling the process controller to initiate feeding of the culture or to increase the existing feed rate. Thereafter, an increasing signal strength indicates that butanol production is increasing and therefore, so is the metabolic rate of the culture. The process control would then increase the feed rate incrementally while continuing to monitor the signal strength of the enzyme. If the signal strength continues to increase, the process controller can continue to increase the feed rate so long as the rate of change of the signal strength of the signal enzyme is increasing. If a decrease in the rate of change for the signal strength of the signal enzyme is noted, the process controller will reduce the feed rate in order not to over feed the culture and cause substrate inhibition and a reduction in butanol production rate. By continued monitoring of the signal enzyme signal and adjusting of the feed rate to reflect the information provided by the signal enzyme, the culture will be place in a state of maximum butanol productivity.

The alternative to the batch-fed process is the continuous batch processes, wherein typically, fermentation broth is simultaneously removed from the fermentor and fresh nutrients or water is added to maintain fermentor volume and desired cell density. Since a continuous fermentation process represents a steady state it can also be monitored and controlled through the use of one or more signal enzymes. Any decrease or increase in signal strength represents a deviation away from the preexisting steady state and depending upon the desired fermentation parameters, such signaling may indicate to the operator or process controller that it is time to adjust the fermentation conditions. The requirement for the continuous removal of fermentation broth in maintaining a steady state provides a ready means to employ in-line measurements of signal enzymes monitoring.

Signal enzymes can also be used for monitoring catabolite repression in a fermentative or synthetic pathway. Some enzymes are sensitive to the concentration of catabolite present, wherein the catabolite is able to bind to the operon for the enzyme and block the transcription of the gene. As catabolite concentration increases the rate of gene transcription for the enzyme decreases. With the use of a signal enzyme construct that utilizes the same transcription regulatory nucleotide sequence, signal strength of the signal enzyme will decline proportionally. When the fermentation process controller detects a drop in the signal strength of the signal enzyme, the process control can take action to counter the accumulation of the repressive catabolite. For example, if the catabolite is a target that is secreted into the media, the process controller can initiate the removal of the target from the culture media. If the catabolite is an intermediary, the intracellular concentration of the repressor can be reduced by increasing the total volume of the culture through the addition of water or fresh culture media.

For organisms that have different inducible fermentative or synthetic pathways the use of a signal enzyme can indicate whether the pathway is active and also indicate the strength of the activity, thereby providing the opportunity to adjust the culture conditions. For example, with C. acetobutylicum, in the acidogenic phase of a batch culture, if a signal enzyme along the solventogenic pathway starts indicating activity along that pathway, the operator or process controller can if desire, add pyruvate to the culture media as a substrate. This induces the expression of acidogenic enzymes thereby prolonging the acidogenic phase. (Junelles A. M. et al. Effect of pyruvate on glucose metabolism in Clostridium acetobutylicum. Biochimie. 69:1183-1190, 1987.) This could be done to provide more organic acids for later reassimulation and conversion of solvents with increased yields.

Similarly, if temperature or pH is found to influence the productivity of a particular fermentative or synthetic pathway, then the use of a signal enzyme could be used to maximize productivity. For example, if a particular strain of C. acetobutylicum, is found to produce more organic acids at one temperature, but a greater concentration of butanol relative to the other solvents at another temperature, then the use of a signal enzyme could indicate when the solventogenic shift has occurred so that the temperature of the culture can be adjusted in a timely manner for maximum butanol productivity.

### 6. Systems and Plants

6.1 Culture Containers

Fermentors for use in the batch fermentation of C. acetobutylicum are well known in the art. (Beesch, S. C. Acetone-butanol fermentation of sugars. Eng. Proc. Dev. 44:1677-1682, 1952; Beesch, S. C. Acetone-butanol fermentation of starches. Appl. Microbiol. 1:85-96, 1953; Killeffer, D. H. Butanol and acetone from corn. A description of the fermentation process. Ind. Eng. Chem. 19:46-50, 1927; MuCutchan W. N., and Hickey, R. J. The butanol-acetone fermentations. Ind. Ferment. 1:347-388, 1954.) Typically, the fermentors to be used have capacities of 50,000 to 200,000 gallons and are without mechanical agitation systems. The mixing of the fermentor contents is facilitated by the sparging of sterile carbon dioxide that also serves to prevent contamination of the culture through the maintenance of positive pressure within the fermentor.

Batch-feed fermentation processes may also be used with C. acetobutylicum fermentations. In certain embodiments, commercially valuable quantities of target products are produced in fermentors with 50,000 l to 200,000 l capacity. In still further embodiments, commercially valuable quantities of target products are produced in fermentors with 200,000 l to 400,000 l capacity. In certain other embodiments, commercially valuable quantities of target products are produced in fermentors with 400,000 l to 800,000 l capacity. In still other embodiments, commercially valuable quantities of targets are produced in fermentors with 800,000 l to 2,000,000 l capacity. In certain embodiments, commercially valuable quantities of targets are produced in fermentors with 2,000,000 l to 4,000,000 l capacity. In other embodiments, commercially valuable quantities of targets are produced in fermentors with 4,000,000 l to 8,000,000 l capacity.

Fermentors for the continuous fermentation of C. acetobutylicum are also known in the art. (U.S. Pat. No. 4,424,275, and U.S. Pat. No. 4,568,643.) Since a high density, steady state culture can be maintained through continuous culturing, with the attendant removal of solvent containing fermentation broth, smaller capacity fermentors can be used. In certain embodiments, commercially valuable quantities of target products are produced in fermentors with 50,000 l to 200,000 l capacity. In still further embodiments, commercially valuable quantities of target products are produced in fermentors with 200,000 l to 400,000 l capacity. In certain other embodiments, commercially valuable quantities of target products are produced in fermentors with 400,000 l to 800,000 l capacity. In still other embodiments, commercially valuable quantities of targets are produced in fermentors with 800,000 l to 2,000,000 l capacity. In certain embodiments, commercially valuable quantities of targets are produced in fermentors with 2,000,000 l to 4,000,000 l capacity. In other embodiments, commercially valuable quantities of targets are produced in fermentors with 4,000,000 l to 8,000,000 l capacity.

The fermentation processes, above, can also utilize immobilized cells as disclosed in WO 81/01012. Immobilization creates cell-free fermentation broth simplifying product recovery and may increase the cell density thereby increasing the production rate of solvents.

6.2 Electronics for Measuring Light

PMT and CCD detection modules are commercially available and can be used off the shelf without extensive modifications as described above. They can be used in conjunction with filters or other spectrographic devices to analyze specific wavelengths. Additionally, fiber optic assemblies are also commercially available to convert photons to an electronic signal.

6.3 Informatics/Software

Accordingly, the systems of this invention can include one or more computers that comprise code that accesses data representing the intensity of the reporter signal either at a single time point, at multiple time points, or continuously over a time period, and code that executes an algorithm that transforms the data into information about the state of one or more biochemical pathways in the culture. The operator or process controller may use this information to regulate culture conditions to increase, maintain or slow down the level of product production.

6.4 Apparatus for Changing Culture Conditions in Response to Signals from Computer

Apparatus for adjusting or changing culture condition are well known in the art and include solenoid actuated or other valves placed on pressurized lines, and the use of pumps for unpressurized fluids. Typically, feed lines, such as those containing glucose or ammonia are maintained under pressure, as are gas lines such as air, nitrogen, or carbon dioxide Additionally, utility lines such as chilled or hot water or steam are pressurized. Upon the issuance of a signal from the process controller, the solenoid is activated and the valve position changes to either open or close the line. In this way, additionally nutrient solution can be added to a fermentor or hot water directed to the fermentor jacket to increase the temperature of the culture.

Unpressurized lines typically comprise specialized, non-bulk, fed components and rely on pumps for the transfer of liquids. Often peristaltic pumps are used in combination with sterile silicon rubber or other pliable tubing. For small quantities of liquids, worm drives can used to meter liquids from syringes. Typically, a process controller will energize or deenergize an electrical circuit thereby turning on or off an electrical pump. In a similar manner, the process controller can direct a pump to remove fermentation broth from a fermentor.

Other fermentation parameters that can be controlled by the process controller include aeration rate, agitation rate and internal atmospheric pressure of the fermentor.

6.5 Means for Harvesting Product

Numerous means are available for the isolation of solvents from fermentation broth including continuous extraction with solvents (U.S. Pat. No. 4,424,275 and U.S. Pat. No. 4,568,643), the use of fluorocarbons (U.S. Pat. No. 4,777,135), the use of absorbent material (U.S. Pat. No. 4,520,104), the use of a pervaporation membrane (U.S. Pat. No. 5,755,967), and the use of a stripping gas (U.S. patent application Ser. No. 10/945,551).

One embodiment of this invention uses a vapor compression distillation system. (U.S. Pat. Nos. 4,671,856, 4,769,113, 4,869,067, 4,902,197, 4,919,592, 4,978,429, 5,597,453, and 5,968,321.) For batch fermentations of C. acetobutylicum the harvesting of solvents contained in the spent fermentation media first requires that the broth be centrifuged to remove cells and particulate matter. The clarified broth is then sent to the distillation system wherein the clarified broth enters a heat exchanger and is preheated by heat transfer from outgoing distilled product and waste fluid. The preheated broth is degassed and fed to a plate-type evaporator/condenser which has counter-flow evaporating and condensing chambers formed alternately between stacked metal plates which are separated by gaskets. The media enters the evaporating chambers where it boils. Heated vapor leaving the evaporating chambers passes through a mesh that removes mist, and is then pressurized by a low pressure compressor. The pressurized vapor is delivered to the condenser chambers, where it condenses as the distilled product, giving up heat to broth in the boiling chambers, and is then discharged from the system. Unvaporized broth containing dissolved solids is likewise collected and discharged from the system.

For continuous cultures of C. acetobutylicum the fermentation broth drawn off the fermentor can be centrifuged to concentrate cells and particulate matter. The concentrated cells and matter can be added back to the fermentor if desired to increase cell density or for further fermentation of partially fermented substrate. Alternately, the clarified fermentation broth can be added back to the fermentor if it contains soluble fermentable substrate. When it is desired to harvest solvents from the media two strategies are available. One is to store clarified fermentation broth until a reasonable quantity is present to initiate a distillation run. Alternatively, clarified fermentation broth can be continuously fed to the vapor distillation system.

Fermentation broth composed of certain butanol containing solvent mixtures may undergo spontaneous phase separate based on specific gravity. The use of a float level indicator can be used to assist in separating the butanol containing solvent layer from the remaining aqueous fraction.

6.6 Biofuel Facility

One embodiment of this invention comprises a biofuel facility. In this embodiment, raw material, in the nature of sugars, dextrins, starches or biomass, is produced onsite. The raw material is then conveyed to the biofuel dock's receiving station where the raw material is segregated and stored according to its nature. As needed, portions of raw material are drawn from storage for preliminary processing into culture substrate media. As culture conditions require, the substrate media is then feed to batch or continuous cultures of fermentative organisms. Once a target concentration of solvents is reached, the fermentation broth is then fed to one or more vapor compression distillation systems where the solvents are separated from the broth. Solvents are fed to an onsite tank farm for temporary storage. The spent broth can be recycled back to the fermentor if fermentable substrate remains. Accumulated microorganisms and unfermented substrate are processed as animal feed or the microorganisms are processed to obtain industrial enzymes. At the shipping dock arriving tank trucks take on solvents, animal feed or enzymes.

6.7 Target Products of Culture

This invention also provides the target products of culture. Compositions produced by the methods of this invention differ from compositions produced by other methods in that at certain stages of production, they harbor traces of their source, that is, compounds from the culture substrate and the microbial organisms that produced them. In the same manner, they lack trace compounds usually found in compositions made using different culture substrates or different strains of C. acetobutylicum or different species of microorganisms. Additionally, compositions of the present invention will also harbor traces that will differ from compositions made in non-fermentative ways.

In one embodiment, the butanol produced by the engineered C. acetobutylicum of the present invention will contain different trace compounds than butanol produced by other C. acetobutylicum strains and fermentative processes because the C. acetobutylicum fermentative process of the present invention will utilize biomass derived from amaranth and/or sweet sorghum, rather than rely on hexoses or corn steep liquor.

6.7.1 Food Grade Aroma and Flavor Extractants and Cosmetic and Pharmaceutical Processing Aids

In one embodiment, the targets of this invention are useful as aroma and flavor extractants or in cosmetic and pharmaceutical processing. One such aroma and flavor extractant and processing aid is butanol. Butanol compositions of this invention differ from butanol compositions derived from petroleum sources. The butanol compositions of this invention comprise distillates from C. acetobutylicum fermentation broth and therefore may contain co-distillates of fermentative origin such as low molecular weight alcohols, aldehydes or esters. The petroleum derived butanol is made primarily by way of hydrogenation of butyraldehyde made through the Oxo process, in which syngas (carbon monoxide and hydrogen) is reacted with propylene. This process produces n-butanol and isobutanol in the range of 8:1 to 10:1 ratios. Therefore, the primary trace contaminant of petroleum derived butanol is expected to be isobutanol. The butanol of this invention can be differentiated from petroleum derived butanol on the basis of the trace composition that can be distinguished through the use of a gas chromatograph (GC) or a GC coupled to a mass spectrometer (MS).

### 7. Business Methods

In one embodiment of this invention, a joint venture is formed between a biotechnology company and an oil refining company. The biotechnology company possesses proprietary bioengineered bacterial strains capable of fermenting biomass into solvents. These solvents have uses as fuel or fuel additives. The oil refining company possesses expertise in petrochemical engineering and also engages in the production of finished petrochemical products for use as fuels. The biotechnology licenses the use of the proprietary bioengineered bacterial strains to the oil refining company. The oil refining company desires to build biomass fermentation plants. The biotechnology company supplies fermentation and process development expertise to the joint venture while the oil refining company supplies engineering expertise. The oil refining company, further, supports the scale-up and process development experiments of the joint venture. The oil refining company purchases the solvents produced by the joint venture biomass fermentation plants and from these proceeds the biotechnology company receives a royalty.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCE LISTING

SEQ ID NO: 1-Optimized luxA Nucleotide Sequence
ATGAAATTTGGATTATTTTTTCTTAATTTTATAAATAGTACAACTATTCAAGAACAGTCAAT
AGCAAGAATGCAGGAGATTACAGAGTATGTTGATAAGCTAAATTTTGAGCAGATTCTTGTA
TGTGAAAATCATTTTTCAGATAATGGTGTTGTAGGTGCTCCTTTAACTGTTAGTGGTTTTTTA
TTAGGACTTACAGAAAAAATTAAGATAGGTTCATTAAATCATGTAATTACTACACATCATC
CAGTTAGAATAGCAGAAGAGGCTTGCCTTTTAGATCAACTTTCTGAAGGAAGATTTATATT
AGGTTTTAGTGATTGTGAAAGAAAAGATGAGATGCACTTTTTTAATAGACCTGAACAATAT
CAACAACAACTTTTTGAAGAGTGCTATGATATTATAAATGACGCATTAACTACAGGATATT
GTAATCCAAATGGAGATTTTTATAATTTTCCTAAAATTTCAGTAAATCCACATGCTTATACT
CAGAATGGTCCTAGAAAGTATGTTACAGCAACTTCTTGTCATGTAGTTGAATGGGCAGCTA
AGAAGGGTATACCATTAATTTTTAAATGGGATGATAGTAATGAAGTAAAACATGAGTATGC
TAAGAGATATCAAGCAATAGCTGGTGAATATGGAGTTGATCTTGCAGAAATTGATCATCAA
TTAATGATATTAGTTAATTATTCAGAGGATTCTGAAAAAGCTAAGGAAGAGACAAGAGCAT
TTATAAGTGATTATATTTTAGCTATGCACCCTAATGAAAATTTTGAAAAAAAACTTGAGGA
AATAATAACTGAAAATTCAGTTGGTGATTATATGGAGTGCACAACTGCTGCAAAACTTGCA
ATGGAAAAATGTGGAGCTAAAGGTATTCTTTTATCTTTTGAAAGTATGTCAGATTTTACACA
TCAGATTAATGCAATAGATATAGTAAATGATAATATTAAGAAATATCATATGTAA
SEQ ID NO: 2-Optimized LuxA Amino Acid Sequence
MKFGNFLLTYQPPQFSQTEVMKRLVKLGRISEECGFDTVWLLEHHFTEFGLLGNPYVAAAYLL
GATKKLNVGTAAIVLPTAHPVRQLEEVNLLDQMSKGRFRFGICRGLYNKDFRVFGTDMNNSR
ALMECWYKLIRNGMTEGYMEADNEHIKFHKVKVLPTAYSQGGAPIYVVAESASTTEWAAQHG
LPMILSWIINTNEKKAQIELYNEVAQEYGHDIHNIDHCLSYITSVDHDSMKAKEICRNFLGHWY
DSYVNATTIFDDSDKTKGYDFNKGQWRDFVLKGHKNTNRRVDYSYEINPVGTPQECIDIIQTDI
DATGISNICCGFEANGTVDEIISSMKLFQSDVMPFLKEKQRSLLY
SEQ ID NO: 3-Optimized luxB Nucleotide Sequence
ATGAAATTTGGATTATTTTTTCTTAATTTTATAAATAGTACAACTATTCAAGAACAGTCAAT
AGCAAGAATGCAGGAGATTACAGAGTATGTTGATAAGCTAAATTTTGAGCAGATTCTTGTA
TGTGAAAATCATTTTTCAGATAATGGTGTTGTAGGTGCTCCTTTAACTGTTAGTGGTTTTTTA
TTAGGACTTACAGAAAAAATTAAGATAGGTTCATTAAATCATGTAATTACTACACATCATC
CAGTTAGAATAGCAGAAGAGGCTTGCCTTTTAGATCAACTTTCTGAAGGAAGATTTATATT
AGGTTTTAGTGATTGTGAAAGAAAAGATGAGATGCACTTTTTTAATAGACCTGAACAATAT
CAACAACAACTTTTTGAAGAGTGCTATGATATTATAAATGACGCATTAACTACAGGATATT
GTAATCCAAATGGAGATTTTTATAATTTTCCTAAAATTTCAGTAAATCCACATGCTTATACT
CAGAATGGTCCTAGAAAGTATGTTACAGCAACTTCTTGTCATGTAGTTGAATGGGCAGCTA
AGAAGGGTATACCATTAATTTTTAAATGGGATGATAGTAATGAAGTAAAACATGAGTATGC
TAAGAGATATCAAGCAATAGCTGGTGAATATGGAGTTGATCTTGCAGAAATTGATCATCAA
TTAATGATATTAGTTAATTATTCAGAGGATTCTGAAAAAGCTAAGGAAGAGACAAGAGCAT
TTATAAGTGATTATATTTTAGCTATGCACCCTAATGAAAATTTTGAAAAAAAACTTGAGGA
AATAATAACTGAAAATTCAGTTGGTGATTATATGGAGTGCACAACTGCTGCAAAACTTGCA
ATGGAAAAATGTGGAGCTAAAGGTATTCTTTTATCTTTTGAAAGTATGTCAGATTTTACACA
TCAGATTAATGCAATAGATATAGTAAATGATAATATTAAGAAATATCATATGTAA
SEQ ID NO: 4-Optimized LuxB Amino Acid Sequence
MKFGLFFLNFINSTTIQEQSIARMQEITEYVDKLNFEQILVCENHFSDNGVVGAPLTVSGFLLGLT
EKIKIGSLNHVITTHHPVRIAEEACLLDQLSEGRFILGFSDCERKDEMHFFNRPEQYQQQLFEECY
DIINDALTTGYCNPNGDFYNFPKISVNPHAYTQNGPRKYVTATSCHVVEWAAKKGIPLIFKWDD
SNEVKHEYAKRYQAIAGEYGVDLAEIDHQLMILVNYSEDSEKAKEETRAFISDYILAMHPNENF
EKKLEEIITENSVGDYMECTTAAKLAMEKCGAKGILLSFESMSDFTHQINAIDIVNDNIKKYHM
SEQ ID NO: 5-Optimized luxC Nucleotide Sequence
ATGAATAAAAAGATATCATTTATTATAAATGGAAGAGTTGAAATATTTCCTGAGTCAGATG
ATTTAGTACAATCTATAAATTTTGGTGATAATTCTGTTCATCTTCCAGTACTTAATGATTCAC
AGGTTAAGAATATTATAGATTATAATGAGAATAATGAGCTTCAGCTTCATAATATTATAAA
TTTTCTTTATACAGTAGGACAGAGATGGAAGAATGAGGAGTATAGCAGAAGAAGAACTTAT
ATAAGAGATCTTAAGAGATATATGGGTTATAGTGAGGAAATGGCAAAATTAGAAGCTAATT
GGATTTCAATGATATTATGTTCTAAGGGAGGTTTATATGATTTAGTTAAAAATGAATTAGGA
AGTAGACATATTATGGATGAATGGTTACCTCAAGATGAATCATATATAAGAGCATTTCCAA
AAGGTAAAAGTGTACATCTTTTAACAGGAAATGTTCCTTTAAGTGGAGTACTTTCAATTTTA
AGAGCTATACTTACTAAAAATCAGTGCATTATAAAGACATCTAGTACTGATCCATTTACAG
CAAATGCTTTAGCACTTAGTTTTATAGATGTTGATCCTCATCATCCAGTAACTAGATCTTTA
AGTGTTGTATATTGGCAACATCAAGGTGATATTTCACTTGCTAAAGAAATAATGCAACATG
CAGATGTTGTAGTTGCTTGGGGAGGTGAAGATGCAATTAATTGGGCTGTAAAGCACGCACC
TCCAGATATAGATGTTATGAAATTTGGACCTAAAAAGTCTTTTTGTATTATAGATAATCCAG
TAGATTTAGTTAGTGCTGCAACAGGTGCTGCACATGATGTATGCTTTTATGATCAGCAGGCT
TGTTTTTCAACTCAAAATATATATTATATGGGATCACATTATGAAGAATTTAAACTTGCATT
AATTGAAAAACTTAATTTATATGCTCATATACTTCCAAATACAAAGAAAGATTTTGATGAA
AAGGCAGCTTATAGTTTAGTTCAGAAAGAATGTTTATTTGCAGGACTTAAAGTAGAAGTTG
ATGTACATCAAAGATGGATGGTTATTGAATCAAATGCTGGTGTAGAATTAAATCAGCCACT
TGGAAGATGCGTTTATTTACATCATGTAGATAATATAGAGCAAATTTTACCTTATGTTAGAA
AGAATAAAACTCAAACAATATCTGTATTTCCATGGGAAGCAGCTTTAAAGTATAGAGATCT
TTTAGCACTTAAAGGTGCTGAAAGAATTGTTGAGGCAGGAATGAATAATATATTTAGAGTA
GGTGGTGCTCATGATGGAATGAGGCCTTTACAGAGACTTGTTACTTATATAAGTCATGAAA
GACCAAGTCATTATACAGCAAAAGATGTAGCTGTAGAGATTGAGCAAACTAGATTTTTAGA
AGAAGATAAGTTTTTAGTATTTGTTCCTTAA
SEQ ID NO: 6-Optimized LuxC Amino Acid Sequence
MNKKISFIINGRVEIFPESDDLVQSINFGDNSVHLPVLNDSQVKNIIDYNENNELQLHNIINFLYTV
GQRWKNEEYSRRRTYIRDLKRYMGYSEEMAKLEANWISMILCSKGGLYDLVKNELGSRHIMD
EWLPQDESYIRAFPKGKSVHLLTGNVPLSGVLSILRAILTKNQCIIKTSSTDPFTANALALSFIDV
DPHHPVTRSLSVVYWQHQGDISLAKEIMQHADVVVAWGGEDAINWAVKHAPPDIDVMKFGP
KKSFCIIDNPVDLVSAATGAAHDVCFYDQQACFSTQNIYYMGSHYEEFKLALIEKLNLYAHILP
NTKKDFDEKAAYSLVQKECLFAGLKVEVDVHQRWMVIESNAGVELNQPLGRCVYLHHVDNIE
QILPYVRKNKTQTISVFPWEAALKYRDLLALKGAERIVEAGMNNIFRVGGAHDGMRPLQRLVT
YISHERPSHYTAKDVAVEIEQTRFLEEDKFLVFVP
SEQ ID NO: 7-Optimized luxD Nucleotide Sequence
ATGGAAAATAAAAGTAGATATAAGACAATAGATCATGTTATTTGTGTAGAGGAGAATAGA
AAGATACATGTTTGGGAAACTTTACCTAAAGAAAATTCACCAAAAAGAAAAAATACACTTA
TTATAGCATCTGGATTTGCTAGAAGAATGGATCATTTTGCTGGTTTAGCTGAATATTTATCT
CAAAATGGATTTCATGTAATTAGATATGATTCATTACATCATGTTGGTTTAAGTTCAGGAAC
TATAGATGAATTTACAATGTCAATTGGTAAGCAGAGTTTACTTGCAGTAGTTGATTGGTTAA
ATACTAGAAAAATAAATAATCTTGGAATGTTAGCTAGTTCATTATCTGCAAGAATAGCTTA
TGCAAGTCTTTCAGAGATTAATGTATCTTTTCTTATAACAGCTGTTGGTGTAGTTAATTTAA
GATATACTTTAGAAAGAGCACTTGGATTTGATTATCTTAGCCTTCCTATTGATGAATTACCA
GATAATCTTGATTTTGAGGGACATAAGTTAGGTGCTGAAGTATTTGCAAGAGATTGCTTTG
ATTCAGGATGGGAAGATCTTACATCTACTATAAATAGTATGATGCACTTAGATATTCCTTTT
ATAGCTTTTACAGCAAATAATGATGATTGGGTTAAACAAGATGAGGTAATTACTCTTCTTTC
TAGTATAAGAAGTCATCAGTGTAAAATATATTCACTTTTAGGTTCTAGTCATGATCTTGGAG
AAAATTTAGTTGTATTAAGAAATTTTTATCAATCAGTTACAAAGGCTGCAATTGCTATGGAT
AATGGTTGCCTTGATATAGATGTAGATATTATAGAACCATCTTTTGAGCATTTAACTATTGC
AGCTGTTAATGAAAGAAGAATGAAAATAGAAATAGAGAATCAAGTAATTAGTTTAAGTTA
A
SEQ ID NO: 8-Optimized LuxD Amino Acid Sequence
MENKSRYKTIDHVICVEENRKIHVWETLPKENSPKRKNTLIIASGFARRMDHFAGLAEYLSQNG
FHVIRYDSLHHVGLSSGTIDEFTMSIGKQSLLAVVDWLNTRKINNLGMLASSLSARIAYASLSEI
NVSFLITAVGVVNLRYTLERALGFDYLSLPIDELPDNLDFEGHKLGAEVFARDCFDSGWEDLTS
TINSMMHLDIPFIAFTANNDDWVKQDEVITLLSSIRSHQCKIYSLLGSSHDLGENLVVLRNFYQS
VTKAAIAMDNGCLDIDVDIIEPSFEHLTIAAVNERRMKIEIENQVISLS
SEQ ID NO: 9-Optimized luxE Nucleotide Sequence
ATGACATCTTATGTTGATAAACAAGAAATAACTGCAAGTTCAGAGATTGATGATTTAATAT
TTAGTTCAGATCCTCTTGTATGGTCTTATGATGAACAGGAAAAGATTAGAAAAAAGTTAGT
TCTTGATGCTTTTAGACATCATTATAAACATTGTCAAGAGTATAGACATTATTGCCAGGCAC
ATAAAGTAGATGATAATATAACAGAAATTGATGATATACCAGTTTTTCCTACTTCAGTATTT
AAGTTTACAAGATTACTTACTTCAAATGAAAATGAGATTGAATCATGGTTTACAAGTTCAG
GAACTAATGGTTTAAAATCTCAAGTTCCAAGAGATAGACTTAGTATAGAAAGACTTTTAGG
ATCAGTATCTTATGGTATGAAGTATATAGGAAGTTGGTTTGATCATCAAATGGAGTTAGTTA
ATCTTGGTCCTGATAGATTTAATGCTCATAATATTTGGTTTAAATATGTAATGTCACTTGTA
GAACTTTTATATCCAACAAGTTTTACTGTAACAGAAGAGCATATAGATTTTGTTCAGACTTT
AAATAGTCTTGAAAGAATTAAACATCAAGGAAAGGATATATGTTTAATTGGTTCACCTTAT
TTTATATATCTTTTATGCAGATATATGAAAGATAAGAATATTTCTTTTAGTGGAGATAAATC
ACTTTATATAATAACTGGAGGTGGATGGAAATCTTATGAAAAGGAGAGTTTAAAAAGAAAT
GATTTTAATCATCTTTTATTTGATACTTTTAATCTTTCAAATATTAATCAAATAAGAGATATT
TTTAATCAGGTAGAATTAAATACATGTTTTTTTGAGGATGAAATGCAAAGAAAACATGTTC
CACCTTGGGTATATGCAAGGGCTCTTGATCCAGAAACTTTAAAGCCTGTTCCAGATGGTAT
GCCTGGACTTATGTCTTATATGGATGCTTCAAGTACTAGTTATCCAGCTTTTATAGTAACTG
ATGATATTGGTATAATAAGTAGAGAATATGGACAATATCCTGGAGTTTTAGTTGAGATTTT
AAGAAGAGTTAATACAAGAAAACAGAAGGGTTGTGCACTTTCATTAACTGAGGCTTTTGGA
TCTTGA
SEQ ID NO: 10-Optimized LuxE Amino Acid Sequence
MTSYVDKQEITASSEIDDLIFSSDPLVWSYDEQEKIRKKLVLDAFRHHYKHCQEYRHYCQAHK
VDDNITEIDDIPVFPTSVFKFTRLLTSNENEIESWFTSSGTNGLKSQVPRDRLSIERLLGSVSYGM
KYIGSWFDHQMELVNLGPDRFNAHNIWFKYVMSLVELLYPTSFTVTEEHIDFVQTLNSLERIKH
QGKDICLIGSPYFIYLLCRYMKDKNISFSGDKSLYIITGGGWKSYEKESLKRNDFNHLLFDTFNL
SNINQIRDIFNQVELNTCFFEDEMQRKHVPPWVYARALDPETLKPVPDGMPGLMSYMDASSTS
YPAFIVTDDIGIISREYGQYPGVLVEILRRVNTRKQKGCALSLTEAFGS
SEQ ID NO: 11-Optimized LuxCDABE genes separated by gram-positive ribosome binding sites
aattcgaattctcagactcaaatagaacaggattctaaagacttaagagcagctgtagatcgtgattttagtacgatagagccaacattgagaaattatgg
ggcaacggaagcacaacttgaagacgccagagccaaaatacacaagcttaaccaagaacagaggttatacaaatgacagttaatacagaggcacta
ataaacagcctaggcaagtcctaccaagaaatttttgatgaagggctaattccttataggaataagccaagtggttctcctggggtgcctaatatttgtat
tgacatggtgaaagaggggatttttttgtcgtttgaacggaatagtaaaatattaaacgaaattactttaagattgcttagagacgataaagctttgttta
tatttccaaatgaattgccatcaccgttgaagcattctatggataggggatgggttagagaaaatttaggtgatctgattaaatcaataccaccgagacaa
attttaaaaaggcagtttggttggaaagatctatatcgttttacggatgaaatcagtatgcagatttcttatgatttacgtgaacaggttaattcagtgac
tttcttgcttacatcagacgtgagttggtaatttaatatatatacccttcatccttcaagttgctgctttgttggctgctttctctcaccccagtcacata
gttatctatgctcctggggattcgttcacttgccgccgcgctgcaacttgaaatctattgggtatatgctattggtaattatggaaaattgcctgatttat
atataacttaacttgtaaaccagataataatttacatgaatattatcacgtataaaaaaattgcgattcttttaatttgaaatagttcaatttaattgaaa
ctttttattaacaaatcttgttgatgtgaaaattttcgtttgctattttaacagatattgttaaacggagaaggcagcatgttgatgattcactcagccag
actgacagttttaagcggaaaattgcagagtatgatcgcattctgataaaggttacaggtcactcgcaaccagaatttcatctttgtatattttgttttgt
tatttacgttgcagcaagacaaaaatagaagaaacaaatatttatacaacccgtttgcaagagggttaaacagcaatttaagttgaaattgccctattaaa
tggagcatgcggatcctcgactttttaacaaaatatattgataaaaataataggatccgggcccctcgagaggaggatggcaaatatgaataaaaagatat
catttattataaatggaagagttgaaatatttcctgagtcagatgatttagtacaatctataaattttggtgataattctgttcatcttccagtacttaat
gattcacaggttaagaatattatagattataatgagaataatgagcttcagcttcataatattataaattttctttatacagtaggacagagatggaagaa
tgaggagtatagcagaagaagaacttatataagagatcttaagagatatatgggttatagtgaggaaatggcaaaattagaagctaattggatttcaatga
tattatgttctaagggaggtttatatgatttagttaaaaatgaattaggaagtagacatattatggatgaatggttacctcaagatgaatcatatataaga
gcatttccaaaaggtaaaagtgtacatcttttaacaggaaatgttcctttaagtggagtactttcaattttaagagctatacttactaaaaatcagtgcat
tataaagacatctagtactgatccatttacagcaaatgctttagcacttagttttatagatgttgatcctcatcatccagtaactagatctttaagtgttg
tatattggcaacatcaaggtgatatttcacttgctaaagaaataatgcaacatgcagatgttgtagttgcttggggaggtgaagatgcaattaattgggct
gtaaagcacgcacctccagatatagatgttatgaaatttggacctaaaaagtctttttgtattatagataatccagtagatttagttagtgctgcaacagg
tgctgcacatgatgtatgcttttatgatcagcaggcttgtttttcaactcaaaatatatattatatgggatcacattatgaagaatttaaacttgcattaa
ttgaaaaacttaatttatatgctcatatacttccaaatacaaagaaagattttgatgaaaaggcagcttatagtttagttcagaaagaatgtttatttgca
ggacttaaagtagaagttgatgtacatcaaagatggatggttattgaatcaaatgctggtgtagaattaaatcagccacttggaagatgcgtttatttaca
tcatgtagataatatagagcaaattttaccttatgttagaaagaataaaactcaaacaatatctgtatttccatgggaagcagctttaaagtatagagatc
ttttagcacttaaaggtgctgaaagaattgttgaggcaggaatgaataatatatttagagtaggtggtgctcatgatggaatgaggcctttacagagactt
gttacttatataagtcatgaaagaccaagtcattatacagcaaaagatgtagctgtagagattgagcaaactagatttttagaagaagataagtttttagt
atttgttccttaataggaggtaaaagaatatggaaaataaaagtagatataagacaatagatcatgttatttgtgtagaggagaatagaaagatacatgtt
tgggaaactttacctaaagaaaattcaccaaaaagaaaaaatacacttattatagcatctggatttgctagaagaatggatcattttgctggtttagctga
atatttatctcaaaatggatttcatgtaattagatatgattcattacatcatgttggtttaagttcaggaactatagatgaatttacaatgtcaattggta
agcagagtttacttgcagtagttgattggttaaatactagaaaaataaataatcttggaatgttagctagttcattatctgcaagaatagcttatgcaagt
ctttcagagattaatgtatcttttcttataacagctgttggtgtagttaatttaagatatactttagaaagagcacttggatttgattatcttagccttcc
tattgatgaattaccagataatcttgattttgagggacataagttaggtgctgaagtatttgcaagagattgctttgattcaggatgggaagatcttacat
ctactataaatagtatgatgcacttagatattccttttatagcttttacagcaaataatgatgattgggttaaacaagatgaggtaattactcttctttct
agtataagaagtcatcagtgtaaaatatattcacttttaggttctagtcatgatcttggagaaaatttagttgtattaagaaatttttatcaatcagttac
aaaggctgcaattgctatggataatggttgccttgatatagatgtagatattatagaaccatcttttgagcatttaactattgcagctgttaatgaaagaa
gaatgaaaatagaaatagagaatcaagtaattagtttaagttaaaacctataccaatagatttcgagttgcagcgcggcggcaagtgaacgcattcccagg
agcatagataactctgtgactggggtgcgtgaaagcagccaacaaagcagcaacttgaaggatgaagggtatattgggatagatagttaactctatcactc
aaatagaaatatactgcaggcggccgcaggaggactctctatgaaatttggaaattttttacttacatatcaacctccacagtttagtcaaactgaagtta
tgaagagattagtaaaacttggtagaatatcagaggaatgtggatttgatacagtttggttacttgaacatcattttactgagtttggtcttttaggaaat
ccttatgtagcagctgcatatttacttggtgctacaaagaaattaaatgtaggtacagcagctattgttttacctacagcacatcctgttagacagttaga
agaagtaaatcttttagatcaaatgtctaaaggtagatttagatttggaatatgcagaggattatataataaggattttagagtttttggtactgatatga
ataatagtagggctcttatggagtgttggtataaattaattagaaatggaatgacagaaggttatatggaagcagataatgagcatataaagtttcataaa
gtaaaagtacttccaactgcttattcacagggaggtgcacctatttatgtagttgctgaatctgcaagtacaactgaatgggctgcacagcatggattacc
aatgatactttcatggattataaatacaaatgagaagaaagctcaaatagaattatataatgaagtagcacaagagtatggacatgatattcataatatag
atcattgcctttcttatattactagtgttgatcatgattcaatgaaagctaaagaaatatgtagaaattttttaggtcattggtatgattcttatgtaaat
gcaacaactatttttgatgatagtgataaaacaaagggatatgattttaataaaggtcagtggagagattttgttcttaaaggacataagaatactaatag
aagagtagattattcatatgaaataaatcctgttggaactccacaagagtgtattgatataatacaaactgatattgatgctacaggaatatctaatattt
gctgtggatttgaagcaaatggtactgtagatgaaataattagtagtatgaagttatttcagtctgatgttatgccttttcttaaggagaaacaaagaagt
ttactttattagctaaggaggaaaatgaaatgaaatttggattattttttcttaattttataaatagtacaactattcaagaacagtcaatagcaagaatg
caggagattacagagtatgttgataagctaaattttgagcagattcttgtatgtgaaaatcatttttcagataatggtgttgtaggtgctcctttaactgt
tagtggttttttattaggacttacagaaaaaattaagataggttcattaaatcatgtaattactacacatcatccagttagaatagcagaagaggcttgcc
ttttagatcaactttctgaaggaagatttatattaggttttagtgattgtgaaagaaaagatgagatgcacttttttaatagacctgaacaatatcaacaa
caactttttgaagagtgctatgatattataaatgacgcattaactacaggatattgtaatccaaatggagatttttataattttcctaaaatttcagtaaa
tccacatgcttatactcagaatggtcctagaaagtatgttacagcaacttcttgtcatgtagttgaatgggcagctaagaagggtataccattaattttta
aatgggatgatagtaatgaagtaaaacatgagtatgctaagagatatcaagcaatagctggtgaatatggagttgatcttgcagaaattgatcatcaatta
atgatattagttaattattcagaggattctgaaaaagctaaggaagagacaagagcatttataagtgattatattttagctatgcaccctaatgaaaattt
tgaaaaaaaacttgaggaaataataactgaaaattcagttggtgattatatggagtgcacaactgctgcaaaacttgcaatggaaaaatgtggagctaaag
gtattcttttatcttttgaaagtatgtcagattttacacatcagattaatgcaatagatatagtaaatgataatattaagaaatatcatatgtaatatacc
ctatggatttcaaggtgcatcgcgacggcaagggagcgaatccccgggagcatatacccaatagatttcaagttgcagtgcggcggcaagtgaacgcatcc
ccaggagcatagataactatgtgactggggtaagtgaacgcagccaacaaagcagcagcttgaaagatgaagggtatagataacgatgtgaccggggtgcg
tgaacgcagccaacaaagaggcaacttgaaagataacgggtataaaagggtatagcagtcactctgccatatcctttaatattagctgccggctagcagga
ggtaaaacaggtatgacatcttatgttgataaacaagaaataactgcaagttcagagattgatgatttaatatttagttcagatcctcttgtatggtctta
tgatgaacaggaaaagattagaaaaaagttagttcttgatgcttttagacatcattataaacattgtcaagagtatagacattattgccaggcacataaag
tagatgataatataacagaaattgatgatataccagtttttcctacttcagtatttaagtttacaagattacttacttcaaatgaaaatgagattgaatca
tggtttacaagttcaggaactaatggtttaaaatctcaagttccaagagatagacttagtatagaaagacttttaggatcagtatcttatggtatgaagta
tataggaagttggtttgatcatcaaatggagttagttaatcttggtcctgatagatttaatgctcataatatttggtttaaatatgtaatgtcacttgtag
aacttttatatccaacaagttttactgtaacagaagagcatatagattttgttcagactttaaatagtcttgaaagaattaaacatcaaggaaaggatata
tgtttaattggttcaccttattttatatatcttttatgcagatatatgaaagataagaatatttcttttagtggagataaatcactttatataataactgg
aggtggatggaaatcttatgaaaaggagagtttaaaaagaaatgattttaatcatcttttatttgatacttttaatctttcaaatattaatcaaataagag
atatttttaatcaggtagaattaaatacatgtttttttgaggatgaaatgcaaagaaaacatgttccaccttgggtatatgcaagggctcttgatccagaa
actttaaagcctgttccagatggtatgcctggacttatgtcttatatggatgcttcaagtactagttatccagcttttatagtaactgatgatattggtat
aataagtagagaatatggacaatatcctggagttttagttgagattttaagaagagttaatacaagaaaacagaagggttgtgcactttcattaactgagg
cttttggatcttgaatgcatgtcgactctagagcatgctagtttctttggaaagaggagcagtcaaaggctcatttgttcaatgcttttgcgaaacgtttt
gtcgaactctaggcgaaggttctcgactttccccgcatcaggggtatatacaagtaaaaaagctcagggggtaaacctgagcttgggatgttgatttttaa
gtatgagatacatgggcggatttaaataacggagtcagtttggaaatatcaacggtcttttctgctttatcgaggctataagtttcttgcagttttaacca
caaccgcggagagctgccaagtacttgtgacagttttattgccatctctggcgtgactgctgctttacacgatactaaacgttgaaccgtagagggagcaa
cattcaatgcccgcgctaagttcacgaattc

What is claimed is:

1. A recombinant nucleic acid molecule comprising a transcription regulatory nucleotide sequence operatively linked with a nucleotide sequence encoding a self-contained light-emitting reporter, wherein the transcription regulatory nucleotide sequence regulates expression of a gene that signals production of a target product of a fermentative or synthetic pathway in a cell.

2. The molecule of claim 1 wherein the transcription regulatory nucleotide sequence is a bacterial transcription regulatory nucleotide sequence.

3. The molecule of claim 1 wherein the transcription regulatory nucleotide sequence regulates expression of a gene encoding an enzyme along the pathway and changes in expression of the reporter are positively correlated with changes in production of the target product.

4. The molecule of claim 1 wherein the transcription regulatory nucleotide sequence regulates expression of a gene encoding an enzyme along a branch off of the pathway and changes in expression of the reporter are negatively correlated with changes in production of the target product.

5. The molecule of claim 1 wherein expression of the reporter increases or decreases with increasing production of target product.

6. The molecule of claim 1 wherein expression of the reporter increases or decreases with decreasing production of target product.

7. The molecule of claim 1 wherein the pathway is a fermentation pathway.

8. The molecule of claim 1 wherein the target product is an end product.

9. The molecule of claim 9 wherein the end product is acetone, ethanol, or butanol.

10. The molecule of claim 1 wherein the target product is an acid intermediate.

11. The molecule of claim 1 wherein the acid intermediate is acetate, butyrate, or lactate.

12. The molecule of claim 1 wherein the pathway is a substrate utilization pathway selected from gluconeogenesis, glycolysis, Entner-Doudoroff pathway or non-oxidative pentose phosphate pathway.

13. The molecule of claim 1 wherein the gene encodes an enzyme along a pathway leading from acetyl CoA to butanol or a branch of that pathway.

14. The molecule of claim 1 wherein the pathway is an anaerobic pathway.

15. The molecule of claim 1 wherein the bacterium converts hexoses, pentoses or amino acids into acids or alcohols.

16. The molecule of claim 1 wherein the transcription regulatory nucleotide sequence is from Clostridium, E. coli, Z. mobilis, or S. cerevisiae.

17. The molecule of claim 1 wherein the gene encodes butanol dehydrogenase, butyraldehyde dehydrogenase, ethanol dehydrogenase, acid aldehyde dehydrogenase, acetoacetate decarboxylase, butyrate kinase, phosphobutyryltransferase, phosphotransacetylase, acetate kinase, acyl CoA transferase, lactate dehydrogenase, butyl CoA transferase.

18. The molecule of claim 1 wherein the self-contained light-emitting reporter is luminescent.

19. The molecule of claim 18 wherein the luminescent reporter comprises luciferase.

20. The molecule of claim 19 wherein the luciferase is from Coleoptera, Photorhabdus, Vibrio, Gaussia, Diptera, Renilla.

21. The molecule of claim 18 wherein the self-contained light-emitting reporter comprises a fluorescent reporter.

22. The molecule of claim 21 wherein the fluorescent reporter comprises green fluorescent protein (“GFP”).

23. The molecule of claim 18 wherein the self-contained light-emitting reporter comprises a phosphorescent reporter.

24. A cell comprising a self-contained reporter construct that indicates when a synthetic or fermentative pathway has been induced or inhibited so as to affect the concentration of an target product of the pathway.

25. A cell comprising a recombinant nucleic acid molecule comprising a transcription regulatory nucleotide sequence operatively linked with a nucleotide sequence encoding a self-contained light-emitting reporter, wherein the transcription regulatory nucleotide sequence regulates expression of a gene that signals production of a target product of a fermentative or synthetic pathway in the cell.

26. The cell of claim 25 that is a bacterial cell.

27. The cell of claim 25 that is Clostridium, E. coli., Z. mobilis or S. cerevisiae.

28. The cell of claim 25 wherein the target product of the pathway in the cell is an end product.

29. The cell of claim 28 wherein the end product of the pathway in the cell is butanol.

30. The cell of claim 25 wherein the gene encodes butanol dehydrogenase, butyraldehyde dehydrogenase, ethanol dehydrogenase, acid aldehyde dehydrogenase, acetoacetate decarboxylase, butyrate kinase, phosphobutyryltransferase, phosphotransacetylase, acetate kinase, acyl CoA transferase, lactate dehydrogenase, or butyl CoA transferase.

31. The cell of claim 30 further comprising a transcription regulatory nucleotide sequence operatively linked with a nucleotide sequence encoding a self-contained light-emitting reporter, wherein the transcription regulatory nucleotide sequence regulates expression of butyraldehyde dehydrogenase

32. A culture comprising cells that produce a target product of a synthetic or fermentative pathway in commercially valuable quantities and a light emitting reporter.

33. A method comprising:
(a) culturing cells that comprise a recombinant nucleic acid molecule comprising a transcription regulatory nucleotide sequence operatively linked to a nucleotide sequence encoding a light-emitting reporter, wherein the transcription regulatory nucleotide sequence regulates expression of a gene that signals the production of a target product of a fermentative or synthetic pathway in the cell, whereby emission of light by the reporter signals production of the target product;
(b) measuring the light emitted from the reporter in the culture; and
(c) changing culture conditions to adjust production of the target product based on the production signaled by the emitted light.

34. The method of claim 33 wherein the light-emitting reporter is self-contained.

35. The method of claim 33 wherein the target product is an end product.

36. The method of claim 33 wherein the target product is an acid intermediate.

37. The method of claim 33 comprising measuring emitted light in real time.

38. The method of claim 33 wherein the emitted light increases or decreases with increasing production of target product.

39. The method of claim 33 wherein the emitted light increases or decreases with decreasing production of target product.

40. The method of claim 33 wherein the cells are cultured in a culture container comprising a window and the light is measured through the window.

41. The method of claim 33 wherein the cells are cultured in a culture container comprising at least one light sensor within the culture that can sense the emitted light and directly or remotely signal a detector.

42. The method of claim 33 wherein the cells are cultured in a culture container comprising a device that continuously flows culture fluid over a light sensor that senses the emitted light in the flow.

43. The method of claim 33 wherein, if target product production decreases, changing culture conditions comprises remove target product, add nutrients, dilute the culture, remove cells. (synthetic pathways are catabolic, fermentation are metabolic or anabolic)

44. A method comprising:
(a) culturing a recombinant cell under culture conditions to produce a target product, wherein the cell comprises a reporter construct that produces a light-based signal, the intensity of which indicates the level of production of the target product;
(b) monitoring continuously over time the intensity of the signal in the culture at a plurality of different times to indicate the level of production of the target product at those times; and
(c) altering the culture conditions in response to changes in target product production to set target product production to a desired level.

45. Software comprising:
code that receives information about the state of a cell or a cell culture,
code that determines whether and how culture conditions should be changed to optimize target production and
code that transmits instructions on changing the culture conditions

46. The software of claim 45 wherein the code determines the state of the cell or cell culture.

47. A system comprising:
a) a container for culturing cells,
b) a photon detector for detecting light in a cell culture in the container; and
c) a computer controlled apparatus changes culture conditions in response to light detected by the detector.

48. The system of claim 47 further comprising a device that converts photons to electrons and electrons to photons.

49. The system of claim 47 further comprising the fermentation chamber comprises at least one window, or at least one light sensor within the culture that can directly or remotely signal a detector, or comprising sampling the culture, a continuous flow detector, whereby the culture fluid is passed over a detector/sensor that measures light.

50. The system of claim 47 further comprising a computer controlled apparatus that removes a target product from the container in response to signal from the computer indicating an amount of production of the target product.

51. A composition comprising substantially of butanol, and containing trace components from amaranth, or sweet sorghum, or both, and substantially free of petroleum by-products.

52. A business method comprising:
a) creating a joint venture between at least a first company that produces bioengineered cells that make a biofuel and a second company engaged in oil refining
b) running the joint venture wherein:
i) the first company provides a license to proprietary bioengineered bacterial strains that produce a biofuel;
ii) the second company sponsors research and development at the joint venture directed to biofuel production; and
iii) the second company purchases biofuel produced by the joint venture.
